(19) United States
(12) Patent Application Publication
(10) Pub. No.: US 2020/0113472 A1
(43) Pub. Date: Apr. 16, 2020

(54) APPARATUS AND METHOD FOR CALCULATING A PULSE DEFICIT VALUE

(71) Applicant: ICAHN SCHOOL OF MEDICINE AT MOUNT SINAI, New Yorkj, NY (US)

(72) Inventors: Ya-El MANDEL-PORTNOY, New York, NY (US); Gregor SCHWARTZ, Dresden (DE)

(73) Assignee: ICAHN SCHOOL OF MEDICINE AT MOUNT SINAI, New York, NY (US)

(21) Appl. No.: 16/712,519

(22) Filed: Dec. 12, 2019

Related U.S. Application Data
(63) Continuation of application No. PCT/US2018/037089, filed on Jun. 12, 2018.
Application No. PCT/US2018/037089 is a continuation of application No. PCT/US2017/037029, filed on Jun. 12, 2017.
Application No. PCT/US2017/037029 is a continuation of application No. PCT/US2017/037029, filed on Jun. 12, 2017.

(51) Int. Cl. A61B 5/0452; A61B 5/024; A61B 5/00; G16H 10/60; G16H 50/30

(52) U.S. Cl. CPC A61B 5/0452; A61B 5/02416; A61B 5/044; G16H 10/60; G16H 50/30; A61B 5/0022

(57) ABSTRACT

Systems, apparatuses, software, and methods for calculating a pulse deficit value of a subject, such as a subject afflicted with a hemodynamic disorder. The devices and apparatuses described herein can include a monitor, at least one ECG sensor, and at least one pulse sensor, where the at least one ECG sensor and the at least one pulse sensor are connected to the monitor, where the monitor converts data collected from the at least one ECG sensor into a value representing depolarization cycle rate, where the monitor is configured to calculate the pulse deficit value based on a number of measured points in time where a difference between the value representing depolarization cycle rate and the value representing pulsation rate exceeds a threshold value, which threshold value is calculated as a fraction of a total number of measured points in time, and where the threshold value is indicative of unacceptable pulse deficit.

### CROSS-REFERENCE

This application is a continuation of PCT/US2018/037089 filed on Jun. 12, 2018, which claims the benefit of PCT/US2017/037029 filed on Jun. 12, 2017 under 35 USC § 119, and which is incorporated here entirely by reference.

### STATEMENT AS TO FEDERALLY SPONSORED RESEARCH

This invention was made with the support of the United States government under grant number HL109005 awarded by the National Institutes of Health. The government has certain rights in the invention.

### BACKGROUND

Atrial Fibrillation is the most common cardiac arrhythmia. Currently, approximately 9% of the U.S. population above the age of 65 suffers from Atrial Fibrillation, with present estimates forecasting that 12 million people in the U.S. will be diagnosed with Atrial Fibrillation by the year 2030. Current evaluation and treatment selection for subjects afflicted with Atrial Fibrillation is based on various factors that fail to account for a subject's adverse hemodynamic effects associated with this condition.

### SUMMARY

The present invention relates to systems, apparatuses, software, and methods for calculating a pulse deficit value of a subject, particularly a subject afflicted with a hemodynamic disorder.

In one aspect, disclosed herein is an apparatus for calculating a pulse deficit value of a subject, including a monitor, at least one electrocardiogram (ECG) sensor, and at least one pulse sensor, where the at least one ECG sensor and the at least one pulse sensor are connected to the monitor, where the monitor is configured to convert data collected from the at least one ECG sensor into a value representing depolarization cycle rate, where the monitor is configured to convert data collected from the at least one pulse sensor into a value representing pulsation rate, and where the monitor is configured to calculate the pulse deficit value based on a number of measured points in time where a difference between the value representing depolarization cycle rate and the value representing pulsation rate exceeds a threshold value, which threshold value is calculated as a fraction of a total number of measured points in time, and where the threshold value is indicative of unacceptable pulse deficit. In some embodiments, a pulse deficit (e.g., an unacceptable pulse deficit) is determined to be present when any difference exists between a number of heart-beats and a number of peripheral pulsations over a period of time. In some embodiments, a pulse deficit is determined to be present when a difference between a number of heart-beats and a number of peripheral pulsations exceeds a threshold value. In some embodiments, a threshold value is equal to 1. In some embodiments, a threshold is equal to 2, 3, 4, 5, 6, 7, 8, 9, or 10 or more. In some embodiments, a pulse deficit is determined to be present when a difference exists between a number of heart beats and a number of peripheral pulsations over a period of time and an additional parameter is present (or multiple additional parameters are present). For example, in some embodiments, an additional parameter comprises a variation of a number of heart-beats from a baseline, wherein a pulse deficit is determined to be present when a difference exists between a number of heart beats and a number of peripheral pulsations over a period of time and a number of heart-beats varies from a baseline value. In some embodiments, the additional parameter comprises an age of a subject.

In certain embodiments, the systems, apparatuses, software, and methods described herein include and/or utilize one or more of the following features. In some embodiments, the monitor comprises a digital screen for displaying the pulse deficit value. In some embodiments, the at least one ECG sensor comprises a 3-lead ECG sensor. In some embodiments, the 3-lead ECG sensor comprises two electrodes configured to attach to the subject's chest and one electrode configured to attach to a lower limb of the subject. In some embodiments, the at least one pulse sensor comprises a photoplethysmographic pulse sensor. In some embodiments, the photoplethysmographic pulse sensor is a finger clip plethysmograph, a finger cuff plethysmograph, an in-ear plethysmograph, a wrist band plethysmograph, an upper arm plethysmograph, or a chest plethysmograph. In some embodiments, the finger cuff plethysmograph comprises a blood pressure bladder, a light source, a light detector, and a wrist unit, where the light source is configured to illuminate underlying tissue in a finger of the subject, where the light detector is configured to detect changes in light intensity associated with variations in blood volume in the underlying tissue, and where the wrist unit is configured to inflate the blood pressure bladder to transmit pressure from the blood pressure bladder to the underlying tissue. In some embodiments, the at least one pulse sensor comprises a bioimpedance pulse sensor. In some embodiments, the bioimpedance pulse sensor is a thoracic bioimpedance plethysmograph, a wrist band bioimpedance plethysmograph, an upper arm bioimpedance plethysmograph, a lower arm bioimpedance plethysmograph, an upper leg bioimpedance plethysmograph, or a lower leg bioimpedance plethysmograph. In some embodiments, the at least one pulse sensor is configured to detect a gyrocardiography (GCG) signal. In some embodiments, the at least one pulse sensor comprises a gyroscope, an accelerometer, or both. In some embodiments, the gyroscope is configured to measure angular velocity. In some embodiments, the gyroscope is a thoracic gyroscope, a wrist band gyroscope, an upper arm gyroscope, a lower arm gyroscope, an upper leg gyroscope, or a lower leg gyroscope. In some embodiments, the accelerometer is configured to measure acceleration. In some embodiments, the accelerometer is a thoracic accelerometer, a wrist band accelerometer, an upper arm accelerometer, a lower arm accelerometer, an upper leg accelerometer, or a lower leg accelerometer. In some embodiments, the monitor is configured to calculate the pulse deficit value based on a calculation of a distance between a generated histogram of the value representing depolarization cycle rate and a generated histogram of the value representing pulsation rate. In some embodiments, the monitor is configured to calculate the pulse deficit value based on a calculation of a difference between the value representing depolarization cycle rate and the value representing pulsation rate at one or more selected percentiles.

In another aspect, disclosed herein is a method for calculating a pulse deficit value of a subject, including measuring and collecting data from at least one ECG sensor, measuring and collecting data from at least one pulse sensor, converting the data from the at least one ECG sensor into a value representing depolarization cycle rate, converting the data from the at least one pulse sensor into a value representing pulsation rate, and calculating the pulse deficit value based on a number of measured points in time where a difference between the value representing depolarization cycle rate and the value representing pulsation rate exceeds a set threshold value, which threshold value is calculated as a fraction of a total number of measured points in time, and where the set threshold value is indicative of unacceptable pulse deficit.

In various aspects, implementations of the systems, apparatuses, software, and methods described herein include one or more of the following features. In some embodiments, the at least one ECG sensor and the at least one pulse sensor are connected to a monitor. In some embodiments, the monitor converts the data from the at least one ECG sensor into the value representing depolarization cycle rate, converts the data from the at least one pulse sensor into the value representing pulsation rate, and calculates the pulse deficit value based on the value representing depolarization cycle rate and the value representing pulsation rate. In some embodiments, the pulse deficit value is displayed on a digital screen of the monitor. In some embodiments, the at least one ECG sensor includes a 3-lead ECG sensor, and in further embodiments, the 3-lead ECG sensor includes two electrodes configured to attach to the subject's chest and one electrode configured to attach to a lower limb of the subject. In some embodiments, the at least one pulse sensor includes a photoplethysmographic pulse sensor, where the photoplethysmographic pulse sensor is optionally a finger clip plethysmograph, a finger cuff plethysmograph, an in-ear plethysmograph, a wrist band plethysmograph, an upper arm plethysmograph, or a chest plethysmograph. In some embodiments, the finger cuff plethysmograph includes a blood pressure bladder, a light source, a light detector, and a wrist unit, where the light source is configured to illuminate underlying tissue in a finger of the subject, where the light detector is configured to detect changes in light intensity associated with variations in blood volume in the underlying tissue, and where the wrist unit is configured to inflate the blood pressure bladder to transmit pressure from the blood pressure bladder to the underlying tissue. In some embodiments, the at least one pulse sensor includes a bioimpedance pulse sensor, where the bioimpedance pulse sensor is a thoracic bioimpedance plethysmograph, a wrist band bioimpedance plethysmograph, an upper arm bioimpedance plethysmograph, a lower arm bioimpedance plethysmograph, an upper leg bioimpedance plethysmograph, or a lower leg bioimpedance plethysmograph. In some embodiments, the at least one pulse sensor is configured to detect a gyrocardiography (GCG) signal. In some embodiments, the at least one pulse sensor comprises a gyroscope, an accelerometer, or both. In some embodiments, the gyroscope is configured to measure angular velocity. In some embodiments, the gyroscope is a thoracic gyroscope, a wrist band gyroscope, an upper arm gyroscope, a lower arm gyroscope, an upper leg gyroscope, or a lower leg gyroscope. In some embodiments, the accelerometer is configured to measure acceleration. In some embodiments, the accelerometer is a thoracic accelerometer, a wrist band accelerometer, an upper arm accelerometer, a lower arm accelerometer, an upper leg accelerometer, or a lower leg accelerometer. In some embodiments, the method further includes calculating the pulse deficit value based on a calculation of a distance between a generated histogram of the value representing depolarization cycle rate and a generated histogram of the value representing pulsation rate. In some embodiments, the method further includes calculating the pulse deficit value based on a calculation of a difference between the value representing depolarization cycle rate and the value representing pulsation rate at one or more selected percentiles.

Described herein is an apparatus configured to determine a presence of a pulse deficit in a subject, the apparatus comprising: a first sensor configured to determine a number of heart-beat occurrences over a period of time based on an electrical signal generated by a heart and sensed by the first sensor; a second sensor configured to determine a number of peripheral pulsations over the period of time based on a signal sensed by the second sensor; a processor; and a non-transitory computer-readable medium including instructions executable by the processor and configured to cause the processor to: receive the number of heart-beat occurrences over the period of time; receive the number of pulsation occurrences over the period of time; and identify the presence of the pulse deficit which comprises a numerical difference between the number of heart-beat occurrences over the period of time and the number of pulsation occurrences over the period of time. Various representations of the numerical difference are contemplated. For example, in some embodiments, the numerical difference is represented as a fraction or a decimal. In some embodiments, the fraction or decimal comprises the number of pulsation occurrences over the number of heart-beat occurrences over the period of time.

In some embodiments, the apparatus comprises a risk stratification classifier configured to assess the risk of an adverse health event occurring to the subject based on the presence of an unacceptable pulse deficit. In some embodiments, a degree of risk of the adverse event occurring corresponds directly to the degree of the numerical difference between the number of heart-beat occurrences over the period of time and the number of pulsation occurrences over the period of time. In some embodiments, the non-transitory computer-readable medium is further configured to cause the processor to: determine a heart rate histogram and a pulse rate histogram; calculate a cosine distance between the heart rate histogram and the pulse rate histogram; and input the cosine distance into the risk stratification classifier to generate the predicted risk category. In some embodiments, the non-transitory computer-readable medium is further configured to cause the processor to: determine the heart rate and the pulse rate for at least two percentiles for a plurality of time points; calculate delta values between the heart rate and the pulse rate for at least two percentiles; and input the delta values for at least two percentiles into the risk stratification classifier to generate the predicted risk category. In some embodiments, the at least two percentiles comprise about 25%, about 50%, and about 75%. In some embodiments, the second sensor comprises a bioimpedance plethysmograph. In some embodiments, the bioimpedance plethysmograph is configured to provide electrical bioimpedance measurements corresponding to the arrival times of peripheral pulsations that are generated by the heart-beats. In some embodiments, the risk stratification classifier generates the predicted risk category based on input indicative of a pulse deficit. In some embodiments the input comprises the numerical difference between the number of heart-beat occurrences over the period of time and the number of pulsation occurrences over the period of time, a delta value between the heart rate and the pulse rate, a cosine distance between a heart rate histogram and a pulse rate histogram, or any combination thereof. In some embodiments, the bioimpedance plethysmograph is a thoracic bioimpedance plethysmograph, a wrist band bioimpedance plethysmograph, an upper arm bioimpedance plethysmograph, a lower arm bioimpedance plethysmograph, an upper leg bioimpedance plethysmograph, or a lower leg bioimpedance plethysmograph. In some embodiments, the at least one pulse sensor is configured to detect a gyrocardiography (GCG) signal. In some embodiments, the at least one pulse sensor comprises a gyroscope, an accelerometer, or both. In some embodiments, the gyroscope is configured to measure angular velocity. In some embodiments, the gyroscope is a thoracic gyroscope, a wrist band gyroscope, an upper arm gyroscope, a lower arm gyroscope, an upper leg gyroscope, or a lower leg gyroscope. In some embodiments, the accelerometer is configured to measure acceleration. In some embodiments, the accelerometer is a thoracic accelerometer, a wrist band accelerometer, an upper arm accelerometer, a lower arm accelerometer, an upper leg accelerometer, or a lower leg accelerometer. In some embodiments, the at least one pulse sensor comprises a pressure sensor. In some embodiments, the pressure sensor is a piezoelectric sensor. In some embodiments, the first sensor comprises an electrocardiogram (ECG) sensor. In some embodiments, the ECG sensor is a 3-lead ECG sensor. In some embodiments, the second sensor comprises a photoplethysmographic (PPG) pulse sensor. In some embodiments, the PPG sensor is finger clip plethysmograph, a finger cuff plethysmograph, an in-ear plethysmograph, a wrist band plethysmograph, an upper arm plethysmograph, or a chest plethysmograph. In some embodiments, the second sensor comprises a photoplethysmographic (PPG) pulse sensor and a bioimpedance plethysmograph. In some embodiments, the apparatus comprises a display for showing at least one of the heart rate, the pulse rate, the pulse deficit value, and the predicted risk category. In some embodiments, the non-transitory computer-readable medium is further configured to cause the processor to upload sensor data to a cloud-based network. In some embodiments, the non-transitory computer-readable medium is further configured to cause the processor to perform signal filtering on sensor data received from the first sensor, the second sensor, or both. In some embodiments, the apparatus is configured as an integrated hardware system comprising the first sensor, the second sensor, and the processor. In some embodiments, the apparatus is configured as a wearable device for daily monitoring. In some embodiments, the wearable device is adapted for long-term continuous monitoring of a subject. In some embodiments, the wearable device is adapted for continuous monitoring of the subject for at least 1 week, 2 weeks, 3 weeks, 4 weeks, 5 weeks, 6 weeks, 7 weeks, 8 weeks, 9 weeks, 10 weeks, 11 weeks, or at least 12 weeks. In some embodiments, the wearable device comprises a smartwatch, a wrist band, a wrist monitor, an upper arm band, or an upper arm monitor. In some embodiments, the non-transitory computer-readable medium is further configured to cause the processor to generate instructions based on the pulse deficit or predicted risk category. In some embodiments, the instructions comprise medication that is to be avoided based on the pulse deficit value or predicted risk category. In some embodiments, the instructions comprise a personalized therapy regimen for reducing a risk of an adverse event. In some embodiments, the instructions comprise taking medication for a low predicted risk category. In some embodiments, the medication comprises amiodorone or procainamide. In some embodiments, the instructions comprise applying an anti-arrhythmic strategy for a high predicted risk category. In some embodiments, the anti-arrhythmic strategy comprises cardioversion or ablation. In some embodiments, the instructions comprise identification of medication that is to be avoided based on the predicted risk category or pulse deficit. In some embodiments, the instructions comprise increased visitation to a healthcare provider (e.g., cardiologist). In some embodiments, the instructions comprise increased monitoring by a healthcare provider. In some embodiments, the instructions comprise using anti-arrhythmic or rate control medication to adjust the pulse deficit.

Described herein is a system configured to determine a presence of a pulse deficit in a subject, the system comprising: a first sensor configured to determine a number of heart-beat occurrences over a period of time based on an electrical signal generated by a heart and sensed by the first sensor; a second sensor configured to determine a number of peripheral pulsations over the period of time based on a signal sensed by the second sensor; a processor; a network element configured to communicate with a network; and a non-transitory computer-readable medium including instructions executable by the processor and configured to cause the processor to: receive the number of heart-beat occurrences over the period of time; receive the number of pulsation occurrences over the period of time; and identify the presence of the pulse deficit which comprises a numerical difference between the number of heart-beat occurrences over the period of time and the number of pulsation occurrences over the period of time.

In some embodiments, the system comprises a risk stratification classifier configured to assess the risk of an adverse health event occurring to the subject based on the presence of an unacceptable pulse deficit. In some embodiments, a degree of risk of the adverse event occurring corresponds directly to the degree of the numerical difference between the number of heart-beat occurrences over the period of time and the number of pulsation occurrences over the period of time. In some embodiments, the risk stratification classifier generates a predicted risk category indicative of the risk of an adverse health event. In some embodiments, the non-transitory computer-readable medium is further configured to cause the processor to: determine a heart rate histogram and a pulse rate histogram; calculate a cosine distance between the heart rate histogram and the pulse rate histogram; and input the cosine distance into the risk stratification classifier to generate the predicted risk category. In some embodiments, the non-transitory computer-readable medium is further configured to cause the processor to: determine the heart rate and the pulse rate for at least two percentiles for a plurality of time points; calculate delta values between the heart rate and the pulse rate for at least two percentiles; and input the delta values for at least two percentiles into the risk stratification classifier to generate the predicted risk category. In some embodiments, the at least two percentiles comprise about 25%, about 50%, and about 75%. In some embodiments, the second sensor comprises a bioimpedance plethysmograph. In some embodiments, the bioimpedance plethysmograph is configured to provide electrical bioimpedance measurements corresponding to the arrival times of peripheral pulsations that are generated by the heart-beats. In some embodiments, the bioimpedance plethysmograph is a thoracic bioimpedance plethysmograph, a wrist band bioimpedance plethysmograph, an upper arm bioimpedance plethysmograph, a lower arm bioimpedance plethysmograph, an upper leg bioimpedance plethysmograph, or a lower leg bioimpedance plethysmograph. In some embodiments, the first sensor comprises an electrocardiogram (ECG) sensor. In some embodiments, the ECG sensor is a 3-lead ECG sensor. In some embodiments, the second sensor comprises a photoplethysmographic (PPG) pulse sensor. In some embodiments, the PPG sensor is finger clip plethysmograph, a finger cuff plethysmograph, an in-ear plethysmograph, a wrist band plethysmograph, an upper arm plethysmograph, or a chest plethysmograph. In some embodiments, the second sensor comprises a photoplethysmographic (PPG) pulse sensor and a bioimpedance plethysmograph. In some embodiments, the second sensor comprises a gyroscope. In some embodiments, the second sensor further comprises an accelerometer. In some embodiments, the second sensor comprises a pressure sensor. In some embodiments, the pressure sensor is a piezoelectric sensor. In some embodiments, the system further comprises a display for showing at least one of the heart rate, the pulse rate, the pulse deficit value, and the predicted risk category. In some embodiments, the non-transitory computer-readable medium is further configured to cause the processor to upload sensor data to a cloud-based network through the network element. In some embodiments, the non-transitory computer-readable medium is further configured to cause the processor to perform signal filtering on sensor data received from the first sensor, the second sensor, or both. In some embodiments, the system is configured as an integrated hardware system comprising the first sensor, the second sensor, and the processor. In some embodiments, the system is configured as a wearable device. In some embodiments, the wearable device is adapted for long-term continuous monitoring of a subject. In some embodiments, the wearable device is adapted for continuous monitoring of the subject for at least 1 week. In some embodiments, the wearable device comprises a smartwatch, a wrist band, or a wrist monitor. In some embodiments, the non-transitory computer-readable medium is further configured to cause the processor to generate instructions based on the pulse deficit value or predicted risk category. In some embodiments, the instructions comprise a personalized therapy regimen for reducing a risk of an adverse event. In some embodiments, the instructions comprise medication that is to be avoided based on the pulse deficit value or predicted risk category. In some embodiments, the instructions comprise taking medication for a low predicted risk category. In some embodiments, the medication comprises amiodorone or procainamide. In some embodiments, the instructions comprise applying an anti-arrhythmic strategy for a high predicted risk category. In some embodiments, the anti-arrhythmic strategy comprises cardioversion or cardiac ablation.

Provided herein is a computer-implemented method for determining a presence of a pulse deficit in a subject, the method comprising: determining a number of heart-beat occurrences over a period of time based on an electrical signal generated by a heart and sensed by a first sensor; determining a number of peripheral pulsations over the period of time based on a signal sensed by a second sensor; and identifying the presence of the pulse deficit which comprises a numerical difference between the number of heart-beat occurrences over the period of time and the number of pulsation occurrences over the period of time.

In some embodiments, the method further comprises providing a risk stratification classifier configured to assess the risk of an adverse health event occurring to the subject based on the presence of an unacceptable pulse deficit. In some embodiments, a degree of risk of the adverse event occurring corresponds directly to the degree of the numerical difference between the number of heart-beat occurrences over the period of time and the number of pulsation occurrences over the period of time. In some embodiments, the risk stratification classifier generates a predicted risk category indicative of the risk of an adverse health event. In some embodiments, the method further comprises: determining a heart rate histogram and a pulse rate histogram; calculating a cosine distance between the heart rate histogram and the pulse rate histogram; and inputting the cosine distance into the risk stratification classifier to generate the predicted risk category. In some embodiments, the method further comprises: determining the heart rate and the pulse rate for at least two percentiles for a plurality of time points; calculating delta values between the heart rate and the pulse rate for at least two percentiles; and inputting the delta values for at least two percentiles into the risk stratification classifier to generate the predicted risk category. In some embodiments, the at least two percentiles comprise about 25%, about 50%, and about 75%. In some embodiments, the second sensor comprises a bioimpedance plethysmograph. In some embodiments, the bioimpedance plethysmograph is configured to provide electrical bioimpedance measurements corresponding to the arrival times of peripheral pulsations that are generated by the heart-beats. In some embodiments, the bioimpedance plethysmograph is a thoracic bioimpedance plethysmograph, a wrist band bioimpedance plethysmograph, an upper arm bioimpedance plethysmograph, a lower arm bioimpedance plethysmograph, an upper leg bioimpedance plethysmograph, or a lower leg bioimpedance plethysmograph. In some embodiments, the first sensor comprises an electrocardiogram (ECG) sensor. In some embodiments, the ECG sensor is a 3-lead ECG sensor. In some embodiments, the second sensor comprises a photoplethysmographic (PPG) pulse sensor. In some embodiments, the PPG sensor is finger clip plethysmograph, a finger cuff plethysmograph, an in-ear plethysmograph, a wrist band plethysmograph, an upper arm plethysmograph, or a chest plethysmograph. In some embodiments, the second sensor comprises a photoplethysmographic (PPG) pulse sensor and a bioimpedance plethysmograph. In some embodiments, the second sensor comprises a gyroscope. In some embodiments, the second sensor further comprises an accelerometer. In some embodiments, the second sensor comprises a pressure sensor. In some embodiments, the pressure sensor is a piezoelectric sensor. In some embodiments, the method further comprises showing at least one of the heart rate, the pulse rate, the pulse deficit value, and the predicted risk category on a display. In some embodiments, the method further comprises uploading sensor data from the first and second sensors to a cloud-based network. In some embodiments, the method further comprises filtering sensor data received from the first sensor, the second sensor, or both. In some embodiments, the method is used for long-term continuous monitoring of a subject. In some embodiments, the subject is monitored for at least 1 week. In some embodiments, the method further comprises generating instructions based on the pulse deficit value or predicted risk category. In some embodiments, the instructions comprise a personalized therapy regimen for reducing a risk of an adverse event. In some embodiments, the instructions comprise medication that is to be avoided based on the pulse deficit value or predicted risk category. In some embodiments, the instructions comprise taking medication for a low predicted risk category. In some embodiments, the medication comprises amiodorone or procainamide. In some embodiments, the instructions comprise applying an anti-arrhythmic strategy for a high predicted risk category. In some embodiments, the anti-arrhythmic strategy comprises cardioversion or cardiac ablation.

Provided herein is non-transitory computer-readable medium including instructions executable by a processor and configured to cause the processor to: determine a number of heart-beat occurrences over a period of time based on an electrical signal generated by a heart and sensed by a first sensor; determine a number of peripheral pulsations over the period of time based on a signal sensed by a second sensor; and identify the presence of the pulse deficit which comprises a numerical difference between the number of heart-beat occurrences over the period of time and the number of pulsation occurrences over the period of time.

In some embodiments, the non-transitory computer-readable media is further configured to cause the processor to provide a risk stratification classifier configured to assess a risk of an adverse health event occurring to the subject based on the presence of an unacceptable pulse deficit. In some embodiments, a degree of risk of the adverse event occurring corresponds directly to the degree of the numerical difference between the number of heart-beat occurrences over the period of time and the number of pulsation occurrences over the period of time. In some embodiments, the risk stratification classifier generates a predicted risk category indicative of the risk of an adverse health event. In some embodiments, the non-transitory computer-readable medium is further configured to cause the processor to: determine a heart rate histogram and a pulse rate histogram; calculate a cosine distance between the heart rate histogram and the pulse rate histogram; and input the cosine distance into the risk stratification classifier to generate the predicted risk category. In some embodiments, the non-transitory computer-readable medium is further configured to cause the processor to: determine the heart rate and the pulse rate for at least two percentiles for a plurality of time points; calculate delta values between the heart rate and the pulse rate for at least two percentiles; and input the delta values for at least two percentiles into the risk stratification classifier to generate the predicted risk category. In some embodiments, the at least two percentiles comprise about 25%, about 50%, and about 75%. In some embodiments, the second sensor comprises a bioimpedance plethysmograph. In some embodiments, the bioimpedance plethysmograph is configured to provide electrical bioimpedance measurements corresponding to the arrival times of peripheral pulsations that are generated by the heart-beats. In some embodiments, the bioimpedance plethysmograph is a thoracic bioimpedance plethysmograph, a wrist band bioimpedance plethysmograph, an upper arm bioimpedance plethysmograph, a lower arm bioimpedance plethysmograph, an upper leg bioimpedance plethysmograph, or a lower leg bioimpedance plethysmograph. In some embodiments, the first sensor comprises an electrocardiogram (ECG) sensor. In some embodiments, the ECG sensor is a 3-lead ECG sensor. In some embodiments, the second sensor comprises a photoplethysmographic (PPG) pulse sensor. In some embodiments, the PPG sensor is finger clip plethysmograph, a finger cuff plethysmograph, an in-ear plethysmograph, a wrist band plethysmograph, an upper arm plethysmograph, or a chest plethysmograph. In some embodiments, the second sensor comprises a photoplethysmographic (PPG) pulse sensor and a bioimpedance plethysmograph. In some embodiments, the second sensor comprises a gyroscope. In some embodiments, the second sensor further comprises an accelerometer. In some embodiments, the second sensor comprises a pressure sensor. In some embodiments, the pressure sensor is a piezoelectric sensor. In some embodiments, the non-transitory computer-readable medium is further configured to cause the processor to show at least one of the heart rate, the pulse rate, the pulse deficit value, and the predicted risk category through a display. In some embodiments, the non-transitory computer-readable medium is further configured to cause the processor to upload sensor data to a cloud-based network. In some embodiments, the non-transitory computer-readable medium is further configured to cause the processor to perform signal filtering on sensor data received from the first sensor, the second sensor, or both. In some embodiments, the non-transitory computer-readable media is further configured to cause the processor to perform long-term continuous monitoring of a subject. In some embodiments, the monitoring of the subject comprises at least 1 week. In some embodiments, the non-transitory computer-readable medium is further configured to cause the processor to generate instructions based on the pulse deficit value or predicted risk category. In some embodiments, the instructions comprise a personalized therapy regimen for reducing a risk of an adverse event. In some embodiments, the instructions comprise medication that is to be avoided based on the pulse deficit value or predicted risk category. In some embodiments, the instructions comprise taking medication for a low predicted risk category. In some embodiments, the medication comprises amiodorone or procainamide. In some embodiments, the instructions comprise applying an anti-arrhythmic strategy for a high predicted risk category. In some embodiments, the anti-arrhythmic strategy comprises cardioversion or cardiac ablation.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic diagram of an embodiment of the apparatus of the present disclosure;

FIG. 2 shows a schematic diagram of another embodiment of the apparatus of the present disclosure;

FIGS. 3A and 3B show schematic diagrams of another embodiment of the apparatus of the present disclosure;

FIG. 4 shows a schematic diagram of another embodiment of the apparatus worn by a user;

FIG. 5 shows a schematic diagram of another embodiment of the apparatus integrated with a Holter monitor;

FIG. 6 shows a flow diagram of an embodiment of a method of determining a pulse deficit value; and

FIG. 7 shows an exemplary embodiment of a system comprising an apparatus as described herein.

### DETAILED DESCRIPTION

Described herein are systems, apparatuses, software, and methods for determining a presence of a pulse deficit in a subject. A pulse deficit is a difference between a number of heart-beats and a number of peripheral pulsations where, in some embodiments, both the heart-beats and peripheral pulsations are measured concurrently over a period of time. In a healthy subject, typically, a pulse deficit does not exist, because in a typical period of time, the number of heart-beats matches the number of peripheral pulsations. In subjects with a pulse deficit, the number of heart-beats that they produce does not match the number of peripheral pulsations that they produce. The mismatch of the heart-beat number of a subject and the peripheral pulsation number of the subject is because there will be one or more heart-beats wherein blood is not ejected from the heart to the peripheral arteries and as such a pulsation is not felt peripherally. In subjects who experience a pulse deficit, their heart goes through a complete diastole and systole cycle, but while the left ventricle generates enough pressure to close the mitral valve (typically causing a heart-sound or heart-beat), the left ventricle does not exert enough force to open (or completely open the aortic valve) and as such a heart-beat can be auscultated (and sensed by, for example, an ECG sensor), enough blood does not leave the left ventricle to generate a peripheral pulsation. Examples of conditions associated with a pulse deficit include Atrial Fibrillation. In Atrial Fibrillation, the atria do not contract properly so that, in certain subjects on certain heart cycles, the left ventricle does not fully fill during diastole. As a result, the left ventricular end diastolic pressure in the sub-optimally filed left ventricle is not enough to force open the aortic valve during systole and eject blood from the heart to the periphery. Accordingly, insufficient blood leaves the left ventricle to generate a detectable peripheral pulsation.

The systems, apparatuses, software, and methods of the present disclosure allow determination of a pulse deficit, which is an important indicator of hemodynamic dysfunction. Hemodynamics is the aspect of cardiovascular physiology encompassing forces that the heart needs to develop in order to circulate blood through the cardiovascular system. Satisfactory blood circulation is a primary condition for supplying a sufficient amount of oxygen to all tissues and, therefore, is associated with cardiovascular health, patient surgery survival, longevity, and quality of life. A significant percentage of all cardiovascular diseases and disorders is related to hemodynamic dysfunction. One such hemodynamic dysfunction is Atrial Fibrillation, which is a cardiac disorder that involves a quivering or irregular heartbeat, i.e., arrhythmia. Subjects afflicted with Atrial Fibrillation suffer from decreased quality of life and higher rates of cardiovascular hospitalization, acute myocardial infarction, heart failure, stroke, blood clotting, cardiovascular death, and other heart-related complications. One consequence of Atrial Fibrillation and the associated irregular heartbeat is apical-radial pulse deficit. Pulse deficit is the difference between the simultaneously counted heart rate (as measured by ECG electrical signal) and the pulse rate at the periphery, including but not limited to the wrist or ankle. Apical-radial pulse deficit occurs when myocardial contraction is intermittently insufficient to propel blood to the periphery with enough force to generate a detectable peripheral pulse. In healthy subjects, there is no difference between the apical heart rate and the peripheral pulse rate. However, as previously mentioned, subjects afflicted with Atrial Fibrillation experience such pulse deficit, which is thought to occur due to either reduction of left ventricular preload or reduced left ventricular contractility.

At the present time, there are two main treatment approaches for subjects afflicted with Atrial Fibrillation. First, there is “rhythm-control” treatment, which involves the restoration and maintenance of sinus rhythm. Second, there is “rate-control” treatment, which involves control of the ventricular rate. Treatment selection is based on consideration of various factors, including a subject's age, history of Atrial Fibrillation occurrences and past treatment failures, past thromboembolic events, e.g., strokes, and severity of symptoms. However, this factored analysis in selecting a treatment approach ignores a subject's adverse hemodynamic effects.

Characterizing adverse hemodynamic changes in subjects afflicted with hemodynamic disorders can lead to more targeted and personalized therapy. One approach for representing the adverse hemodynamic effects in subjects afflicted with hemodynamic disorders is by measuring the severity and magnitude of pulse deficit. Therefore, there exists a need for effective means to measure pulse deficit in subjects afflicted with hemodynamic disorders to stratify such subjects based on pulse deficit severity and magnitude, which is correlated to risk level for adverse events and worsening symptoms. Accordingly, the present systems, apparatuses, software, and methods described herein provide a solution for effectively measuring a pulse deficit for stratifying subjects based on the severity and magnitude of the pulse deficit.

### Devices or Apparatuses for Determining a Presence of a Pulse Deficit in a Subject

In some embodiments, an apparatus as described herein includes: (1) a first sensor that senses an electric signal associated with the heart indicative of a heart cycle (e.g., diastole and systole) which is a proxy for a heart-beat, (2) a second sensor that senses a peripheral pulsation, (3) a processor configured to receive a signal from the first sensor indicating the occurrence of a heart-beat and a signal from the second sensor indicating the occurrence of a peripheral pulsation, said processor further configured to integrate the received signals and determine if a pulse deficit is present. In some embodiments, the processor is further configured to notify either a subject having the pulse deficit that is determined to be present or a health care provider of said subject of the occurrence of the pulse deficit.

In some embodiments, the apparatus as describe herein takes the form of a stand-alone monitor, a wearable apparatus, or an element or platform technology to be incorporated into an existing monitor or physiological measurement system. In some embodiments, the wearable apparatus operates wirelessly or via wired connection, and may take a form including but not limited to a watch, cuff, sock, earphone, earbud, patch, sticker, band, or strap. In some embodiments, the existing monitor or physiological measurement system that may incorporate the present invention is a medical monitor or a patient bedside monitor. For example, in some embodiments, the apparatus described herein is incorporated into a system comprising a Holter monitor. In some embodiments, the apparatus as described herein is configured to perform comparison diagnostics to rule out patients for clinical trials.

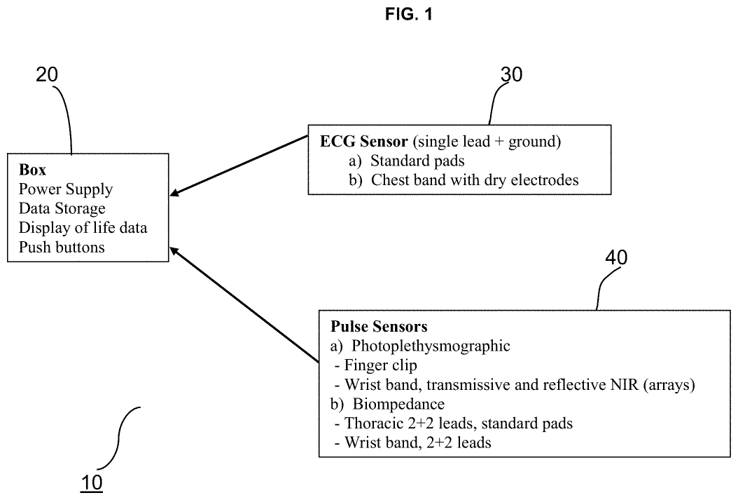

FIG. 1 shows an overview of an apparatus 10 for calculating a pulse deficit value of a subject according to one embodiment of the present invention. Apparatus 10 includes three components: a monitor 20, at least one ECG sensor 30, and at least one pulse sensor 40. In some embodiments, the monitor 20 includes one or more of the following components: a power supply, a microcontroller, data storage 30 capabilities, a display of one or more signal/vital sign data, push buttons to start and stop signal/vital sign data recording, input ports for sensor signals, output ports for probes, a probe driver, a sensor read-out, and Bluetooth or other wireless communication protocol capabilities. In some embodiments, the monitor 20 optionally comprises a display element in an embodiment where the signal/vital sign data is transmitted wirelessly, including by cloud computing, to a recipient. In some embodiments, the at least one ECG sensor 30 includes one or more of the following components: a lead, a ground lead, shielded cables connectable to standard ECG pads, an interchangeable chest band with dry electrodes, a differential amplifier, baseline wandering compensation capabilities, an analog-to-digital converter (ADC), and a feed signal to a microcontroller. In some embodiments, the at least one pulse sensor 40 includes one or more of the following components: a photoplethysmographic pulse sensor, a bioimpedance pulse sensor, a gyroscope and/or accelerometer, and a pressure sensor (e.g. piezoelectric sensor). In some embodiments, the photoplethysmographic pulse sensor includes one or more of the following components: a finger clip plethysmograph with transmissive near-infrared (NIR) light-emitting diode (LED) capabilities, a wrist band plethysmograph with transmissive and reflective NIR LED capabilities, an in-ear plethysmograph, an upper arm plethysmograph, a chest plethysmograph, tunable LED intensity capabilities, multiplexing LEDs, a photodiode (PD), and a feed signal to a microcontroller. In some embodiments, the bioimpedance pulse sensor includes one or more of the following components: a thoracic bioimpedance plethysmograph, a wrist band bioimpedance plethysmograph, an upper arm bioimpedance plethysmograph, a lower arm bioimpedance plethysmograph, an upper leg bioimpedance plethysmograph, a lower leg bioimpedance plethysmograph, shielded cables connectable to standard ECG pads, a variable AC current supply, a voltage signal, an ADC, and a feed signal to a microcontroller. In another embodiment, the photoplethysmographic pulse sensor and the bioimpedance pulse sensor are both incorporated into a wearable device, such as a wrist band or a finger cuff. In some embodiments, any combination of the photoplethysmographic pulse sensor, bioimpedance pulse sensor, gyroscope and/or accelerometer, or pressure sensor is incorporated into a wearable device such as a wrist band or a finger cuff.

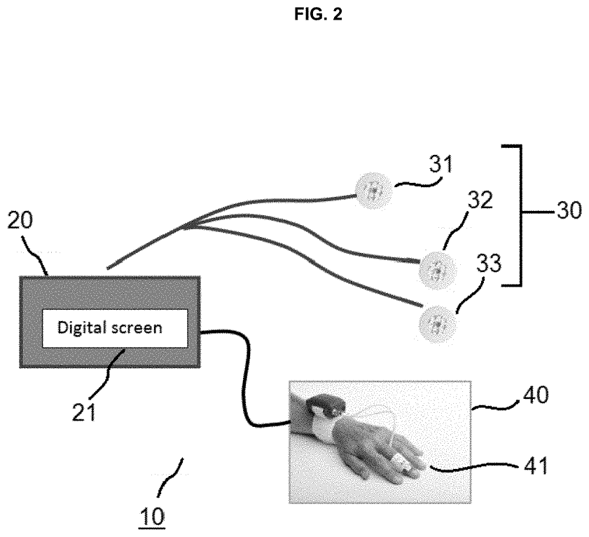

FIG. 2 shows an overview of an apparatus 10 for calculating a pulse deficit value of a subject according to another embodiment of the present invention. The apparatus 10 includes three components: a monitor 20, at least one ECG sensor 30, and at least one pulse sensor 40. In some embodiments, the monitor 20 includes a digital screen 21, an internal drive, and two sets of cable exits leading to the at least one ECG sensor 30 and the at least one pulse sensor 40, respectively. In some embodiments, the digital screen 21 is capable of displaying numerical and/or graphical representations of information. In some embodiments, the graphical representations comprise, but are not limited to, a dashboard display, a bar or color spectrum, or a cartoon face spectrum, such as the Wong-Baker FACES Pain Rating Scale, to indicate the calculated pulse deficit value or related calculated value or measurement.

As shown in FIG. 2, the at least one ECG sensor 30 includes a 3-lead ECG sensor having electrodes 31, 32, and 33. Of electrodes 31, 32, 15 and 33, two of these electrodes are configured to attach to the subject's chest while one of these electrodes is configured to attach to a lower limb of the subject. In some embodiments, the at least one ECG sensor comprises at least three, at least four, at least five, at least six, at least seven, at least eight, at least nine, at least ten electrodes, at least eleven, or at least twelve electrodes. In some embodiments, the at least one ECG sensor comprises at least three, at least four, at least five, at least six, at least seven, at least eight, at least nine, at least ten electrodes, at least eleven, or at least twelve leads.

In some embodiments, the at least one pulse sensor 40 includes a finger cuff plethysmograph 41. In some embodiments, the main components of finger cuff plethysmograph 41 include a blood pressure bladder, a light source, a light detector, and a wrist unit. The light source is configured to illuminate underlying tissue in a finger of the subject. The light detector is configured to detect changes in light intensity associated with variations in blood volume in the underlying tissue. The relevant light with respect to the light source and the light detector may be selected from, but is not limited to, LED light, infrared light, or other acceptable electromagnetic radiation. For example, the light source and the light detector may be configured for transmitting and receiving infrared light, respectively. In some embodiments, the wrist unit is configured to inflate the blood pressure bladder to transmit pressure from the blood pressure bladder to the underlying tissue.

Once the apparatus 10 is powered and connected to a subject (e.g., the at least one ECG sensor 30 is attached to the subject's body and the at least one pulse sensor 40 is attached to the subject's finger), the monitor 20 collects simultaneously recorded data from both the at least one ECG sensor 30 and the at least one pulse sensor 40 over a set time period (e.g., a period of greater than one minute and less than 45 minutes). The monitor 20 collects electrical signals from the at least one ECG sensor 30 and pulsations from the at least one pulse sensor 40. In some embodiments, this simultaneously recorded data is then stored on the internal drive of monitor 20 and is optionally organized with respect to three different parameters: sample time (using 50 Hz to 1 kHz); electrical signal (e.g., depolarization cycle, at each point in time); and pulsation (e.g., change in artery volume), at each point in time. In some embodiments, the sample time comprises at least 50 Hz, at least 60 Hz, at least 70 Hz, at least 80 Hz, at least 90 Hz, at least 100 Hz, at least 150 Hz, at least 200 Hz, at least 250 Hz, at least 300 Hz, at least 350 Hz, at least 400 Hz, at least 450 Hz, at least 500 Hz, at least 550 Hz, at least 600 Hz, at least 650 Hz, at least 700 Hz, at least 750 Hz, at least 800 Hz, at least 850 Hz, at least 900 Hz, at least 950 Hz, or at least 1000 Hz. In some embodiments, the sample time comprises up to 50 Hz, up to 60 Hz, up to 70 Hz, up to 80 Hz, up to 90 Hz, up to 100 Hz, up to 150 Hz, up to 200 Hz, up to 250 Hz, up to 300 Hz, up to 350 Hz, up to 400 Hz, up to 450 Hz, up to 500 Hz, up to 550 Hz, up to 600 Hz, up to 650 Hz, up to 700 Hz, up to 750 Hz, up to 800 Hz, up to 850 Hz, up to 900 Hz, up to 950 Hz, or up to 1000 Hz. In some embodiments, both the electrical signal and pulsation are measured as a time series of beats per minute. In some embodiments, additional recorded data and parameters comprises one or more of blood pressure, oxygen saturation, arterial pressure, or capillary pressure. In some embodiments, the apparatus comprises one or more additional sensors for obtaining additional recorded data and parameters. In some embodiments, the one or more additional sensors include a blood pressure sensor and/or an oximeter (e.g. for pulse oximetry to determine arterial oxygen saturation). In some embodiments, the oximeter is integrated into a PPG sensor such as a finger PPG sensor. In some embodiments, the blood pressure sensor is integrated into a PPG sensor. In some embodiments, the blood pressure sensor is integrated into the monitor.

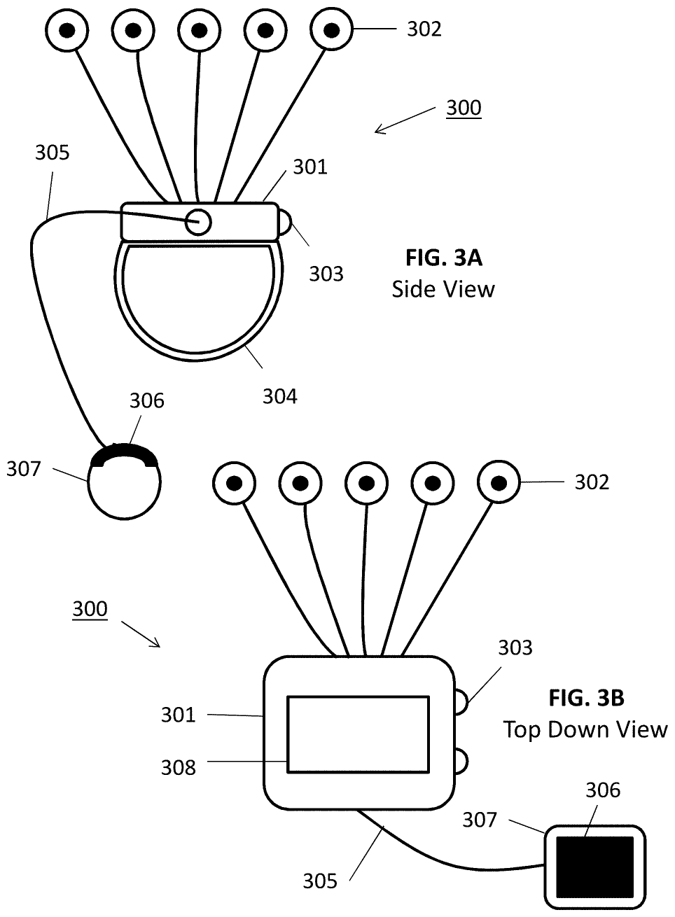

FIG. 3A and FIG. 3B show another embodiment of the apparatus 300 as described herein. FIG. 3A shows a side view of the apparatus. FIG. 3B shows a top down view of the apparatus. In this embodiment, the apparatus comprises a monitor 301. The apparatus comprises a plurality of electrodes 302 for measuring electrical signals from the heart. The apparatus 300 comprises one or more interface elements 303 (e.g. buttons, dials, switches, toggles, wheels, or knobs) allowing a subject to control and/or communicate with the apparatus. In some embodiments, the apparatus 300 is configured as a wearable apparatus. For example, the apparatus shown in FIG. 3A comprises a wrist wrap or band 304 to couple the apparatus 300 to the forearm and/or wrist of the subject. In some embodiments, the apparatus 300 comprises a display 308 for displaying information such as, for example, one or more of sensor data, heart-beat rate, pulsation rate, or pulse rate deficit values. In some embodiments, the apparatus 300 comprises a wired connection 305 to a PPG sensor as described herein such as a finger cuff or clip 307 having a plethysmograph sensor 306. In some embodiments, the apparatus 300 comprises an impedance sensor, a gyroscope and/or accelerometer, or a pressure sensor (e.g. a third sensor). In some embodiments, the impedance sensor, gyroscope and/or accelerometer, or pressure sensor is integrated into the wrist wrap or band 304. In some embodiments, the impedance sensor, gyroscope and/or accelerometer, or pressure sensor is integrated into the finger cuff or clip 307. In some embodiments, the monitor 301 comprises a display such as a digital display screen 308. In some embodiments, the display 308 is a touchscreen. In some embodiments, the display 308 is a light-emitting diode display (LED), a liquid crystal display (LCD), organic light-emitting diode display (OLED), a digital light processing display (DLP), or an electronic paper display (e.g. an electrophoretic display such as E-Ink). In some embodiments, the monitor 301 comprises a digital processing device for performing computational calculations such as analysis of the sensor data. In some embodiments, the monitor 301 comprises one or more sensors such as an impedance sensor, a PPG sensor, a gyroscope and/or accelerometer, or a pressure sensor (e.g., locating the PPG sensor on a monitor located on the wrist instead of on the finger).

In some embodiments, the monitor 301 is in communication with the first sensor 302 and/or the second sensor 306 as shown in FIG. 3A and FIG. 3B. In some embodiments, the monitor 301 comprises wireless connections to the first sensor 302 and/or the second sensor 306. In some embodiments, the apparatus 300 comprises an internal power source such as a battery or battery pack. In some embodiments, the internal power source comprises a rechargeable battery or battery pack. In some embodiments, the apparatus 300 comprises a wired power adaptor to provide power for the apparatus 300 and/or to recharge the internal power source.

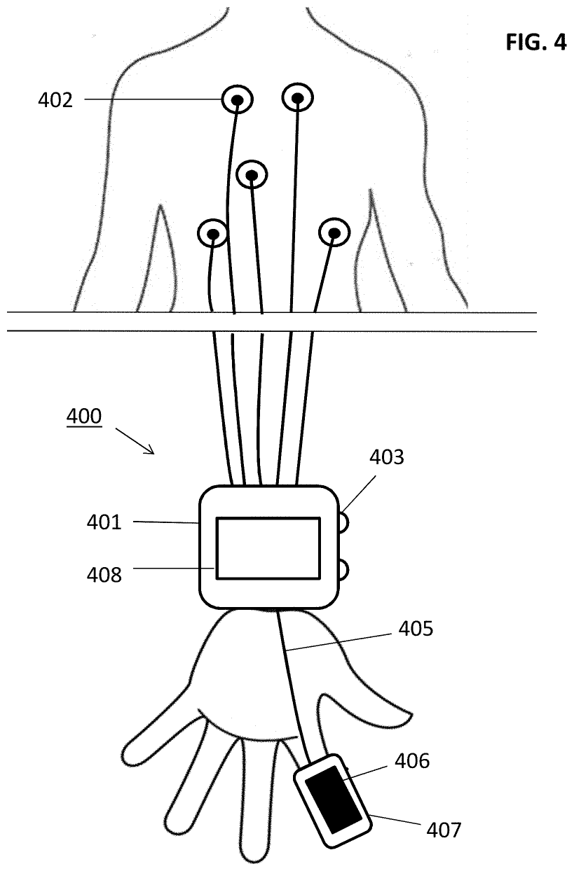

FIG. 4 shows another embodiment of an apparatus 400 described herein when worn or used by the subject. The electrodes 402 are positioned on the subject's torso to allow measurement of electrical signals, which are conveyed through electrical leads connecting the electrodes 402 to the monitor 401. In this embodiment, the monitor 401 is positioned over the subject's wrist/forearm. In some embodiments, the apparatus 400 comprises an impedance sensor, a plethysmograph sensor, a gyroscope and/or accelerometer, a pressure sensor, or any combination thereof (e.g., integrated into the wrist wrap or band of the monitor 401) for measuring the occurrence of a pulsation. As shown in FIG. 4, the monitor 401 comprises two buttons 403 allowing user interaction or input. The monitor 401 also comprises a display for showing information such as, for example, heart-beat rate, pulse rate, and/or pulse deficit value. The monitor 401 is coupled to a finger cuff or clip 407 containing a plethysmograph sensor 406.

In some embodiments, the sensor data from multiple sensors are utilized to calculate a pulsation rate. In some embodiments, sensor data from any combination of a plethysmograph sensor, an impedance sensor, a gyroscope and/or accelerometer, and a pressure sensor is utilized to calculate a pulsation rate. In some embodiments, sensor data from the plethysmograph sensor and the impedance sensor are utilized to calculate a pulsation rate. For example, readings from a plethysmograph sensor can be disrupted or susceptible to noise when the subject is moving. The use of one or more additional sensors allows for a reduction in noise or other disruptions. Accordingly, in some embodiments, when there is a difference in the pulsation rates determined using the plethysmograph and another sensor (e.g., impedance sensor) that exceeds a certain value (e.g. a discrepancy in sensor readings), the apparatus is configured to account for the discrepancy. As an example, the apparatus is configured to discard the sensor data for the period of time during which the discrepancy is detected and utilizes data from another period of time that does not have the discrepancy. In some embodiments, the apparatus is configured to utilize only data from the other sensor (e.g., impedance sensor) to generate a pulsation rate and use it to determine a pulse deficit value when the discrepancy is detected.

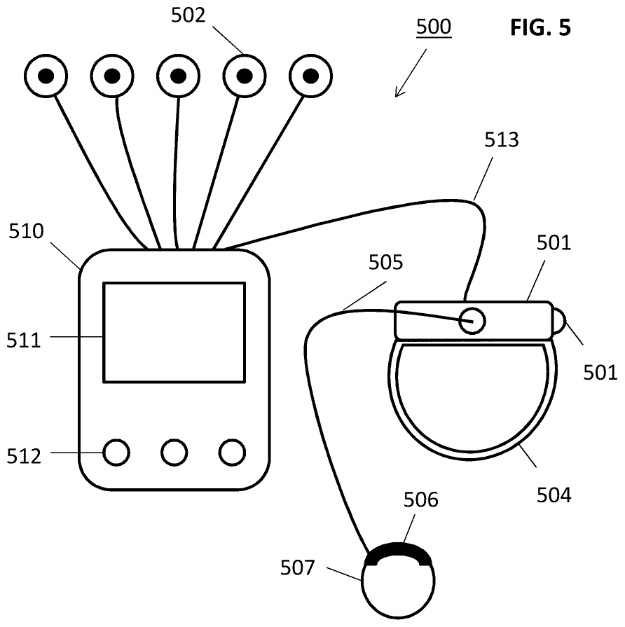

FIG. 5 shows another embodiment of an apparatus 500 described herein when the components of the apparatus 500 such as the monitor 501 and/or PPG sensor 506 are integrated with a Holter monitor 510. In this embodiment, the Holter monitor 510 comprises a plurality of electrodes 502, a display 511, and three buttons 512 for receiving user input or commands. The Holter monitor 510 is coupled to the monitor 501 via a wired connection 513. The monitor 501 comprises a button 501, a wrist cuff or wrap 504, and a wired connection 505 to a finger clip or band 507 with a PPG sensor 506. The monitor 501 is configured to receive sensor data from the PPG sensor 506 and the Holter monitor 510, which provides the electrical signal readings obtained from its electrodes 502. Alternatively, in some embodiments, the monitor and the Holter monitor are in wireless communication without requiring a wired connection.

In some embodiments, the apparatus comprises a non-transitory computer-readable medium comprising instructions executable by the processor. In some embodiments, the instructions executable by the processor are encoded by the software that is integrated into the hardware apparatus described herein.

The apparatuses described herein can be adapted for various applications. In some embodiments, the apparatus is configured to be worn by a subject. In some embodiments, the apparatus comprises a monitor configured to be worn on a wrist or forearm of the subject. In some embodiments, the monitor is configured to be worn on an upper arm of the subject. In some embodiments, the monitor is configured to be worn on the person of the subject via a clip, wrap, or other tool for coupling the monitor to the subject or the clothing of the subject. For example, in some embodiments, the monitor comprises a clip allowing the monitor to be worn on a belt or pants of the subject. In some embodiments, the monitor comprises a neck band for wearing the monitor (e.g. similar to a Holter monitor). In some embodiments, the apparatus is configured as a portable or hand-held device. A hand-held device allows convenient use in the clinic or emergency settings such as in an ambulance. In some embodiments, the apparatus is configured as a stand-alone bedside monitor. In some embodiments, the apparatus is integrated into an existing bedside monitor. For example, in certain embodiments, the apparatus is configured for embedded into an existing bedside monitor as an add-on component (e.g., plugs into the bedside monitor to add signals to be collected). In some embodiments, the apparatus is configured for continuous monitoring of a subject. For example, in some embodiments, the apparatus is configured to monitor a subject for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks.

### Software for Determining a Presence of a Pulse Deficit in a Subject

In some embodiments, software as described herein is configured to receive and integrate sensed heart-related data and peripheral pulse-related data. In some embodiments, software as described herein is configured to determine a number of heart-beats (or number of heart-beat proxies) and/or a number of occurrences of a peripheral pulsation over a period of time. In these embodiments, the software provides a heart-beat rate (also sometimes referred to as a heart rate or depolarization cycle rate) and a peripheral pulsation rate (also sometimes referred to as a pulse rate). In some embodiments, the heart rate and pulse rate (or pulse deficit value) are calculated as a fraction, a ratio, or a decimal of each other (e.g., pulse rate over heart rate). An example of a heart-beat rate as described herein is a depolarization cycle rate detected using an ECG sensor. In these embodiments, the software is configured to determine a difference between a number of heart-beats and a number of occurrences of a peripheral pulsation, wherein the heart-beats and the peripheral pulsations are measured over the same period of time. A period of time over which a number of heart-beats and number of peripheral pulsations is monitored is typically concurrent for both the number of heart-beats and the number of peripheral pulsations. However, in some embodiments, a number of heart-beats and peripheral pulsations are measured in non-concurrent periods of time. In some embodiments, period of time over which a number of heart-beats and/or number of peripheral pulsations is monitored is one week. In some embodiments, a period of time over which a number of heart-beats and/or number of peripheral pulsations is monitored is one day. In some embodiments, a period of time over which a number of heart-beats and/or number of peripheral pulsations is monitored is 12 hours. In some embodiments, a period of time over which a number of heart-beats and/or number of peripheral pulsations is monitored is 6 hours. In some embodiments, a period of time over which a number of heart-beats and/or number of peripheral pulsations is monitored is 3 hours. In some embodiments, a period of time over which a number of heart-beats and/or number of peripheral pulsations is monitored is 2 hours. In some embodiments, a period of time over which a number of heart-beats and/or number of peripheral pulsations is monitored is 1 hour. In some embodiments, a period of time over which a number of heart-beats and/or number of peripheral pulsations is monitored is 30 minutes. In some embodiments, a period of time over which a number of heart-beats and/or number of peripheral pulsations is monitored is 15 minutes. In some embodiments, a period of time over which a number of heart-beats and/or number of peripheral pulsations is monitored is 10 minutes. In some embodiments, a period of time over which a number of heart-beats and/or number of peripheral pulsations is monitored is 5 minutes. In some embodiments, a period of time over which a number of heart-beats and/or number of peripheral pulsations is monitored is 5 minutes. In some embodiments, a period of time over which a number of heart-beats and/or number of peripheral pulsations is monitored is 5 minutes. In some embodiments, a period of time over which a number of heart-beats and/or number of peripheral pulsations is monitored is 3 minutes. In some embodiments, a period of time over which a number of heart-beats and/or number of peripheral pulsations is monitored is 2 minutes. In some embodiments, a period of time over which a number of heart-beats and/or number of peripheral pulsations is monitored is 2 minutes. In some embodiments, a period of time over which a number of heart-beats and/or number of peripheral pulsations is monitored is 1 minute. In some embodiments, a period of time over which a number of heart-beats and/or number of peripheral pulsations is monitored is 30 seconds. In some embodiments, a period of time over which a number of heart-beats and/or number of peripheral pulsations is monitored is 15 seconds. In some embodiments, a period of time over which a number of heart-beats and/or number of peripheral pulsations is monitored is 10 seconds. In some embodiments, a period of time over which a number of heart-beats and/or number of peripheral pulsations is monitored is 5 seconds. In some embodiments, a period of time over which a number of heart-beats and/or number of peripheral pulsations is monitored is 1 second. In some embodiments, a period of time over which a number of heart-beats and/or number of peripheral pulsations is monitored is at least 1 week, at least 6 days, at least 5 days, at least 4 days, at least 3 days, at least 2 days, at least 24 hours, at least 20 hours, at least 16 hours, at least 12 hours, at least 8 hours, at least 4 hours, at least 3 hours, at least 2 hours, at least 60 minutes, at least 50 minutes, at least 40 minutes, at least 30 minutes, at least 25 minutes, at least 20 minutes, at least 15 minutes, at least 10 minutes, at least 5 minutes, at least 4 minutes, at least 3 minutes, at least 2 minutes, at least 60 seconds, at least 50 seconds, at least 40 seconds, at least 30 seconds, at least 25 seconds, at least 20 seconds, at least 15 seconds, at least 10 seconds, at least 5 seconds, or at least 1 second. In some embodiments, a period of time over which a number of heart-beats and/or number of peripheral pulsations is monitored is no more than 1 week, no more than 6 days, no more than 5 days, no more than 4 days, no more than 3 days, no more than 2 days, no more than 24 hours, no more than 20 hours, no more than 16 hours, no more than 12 hours, no more than 8 hours, no more than 4 hours, no more than 3 hours, no more than 2 hours, no more than 60 minutes, no more than 50 minutes, no more than 40 minutes, no more than 30 minutes, no more than 25 minutes, no more than 20 minutes, no more than 15 minutes, no more than 10 minutes, no more than 5 minutes, no more than 4 minutes, no more than 3 minutes, no more than 2 minutes, no more than 60 seconds, no more than 50 seconds, no more than 40 seconds, no more than 30 seconds, no more than 25 seconds, no more than 20 seconds, no more than 15 seconds, no more than 10 seconds, no more than 5 seconds, or no more than 1 second.

It should also be understood that a period of time over which a number of heart-beats and a number of peripheral pulsations is typically the same length for both the measurement of the number heart-beats and the number of peripheral pulsations. However, in some embodiments, a number of heart-beats is measured over a first period of time and a number of peripheral pulsations is measured over a second period of time having a longer duration than the first period of time. Likewise, in some embodiments, a number of heart-beats is measured over a first period of time and a number of peripheral pulsations is measured over a second period of time and the first period of time has a longer duration than the second period of time.

It should also be understood that in some embodiments, a duration over which a number of heart-beats and a number of peripheral pulsations is measured is the duration of a single heart-beat or a single peripheral pulsation. That is, in such embodiments, a pulse deficit is determined to be present with a single heart-beat.

In some embodiments, a software application as described herein is configured to integrate with one or more different sensors, sensing devices, or other software applications. In some embodiments, software as described herein is configured to receive heart-beat related data from a traditional Holter monitor/wireless Holter monitor. In these embodiments, a Holter monitor is either integrated with a device containing the software described herein by, for example, a physical connection including a hardwired connection, or the software is configured to receive a wireless transmission from the Holter monitor. In some embodiments, the heart-beat related data from a Holter monitor comprises data from an ECG sensor. Similarly, in some embodiments, software as described herein is configured to receive pulsation (e.g., peripheral pulse) related data from a pulse sensor such as a PPG sensor. In some embodiments, the software as described herein is configured to receive pulsation related data from a PPG sensor. In some embodiments, software as described herein is configured to receive data from an impedance sensor. In some embodiments, the software is configured to integrate pulsation related data from a PPG sensor and a bioimpedance sensor/pressure sensor/gyroscope and/or accelerometer for calculating a pulsation rate. In some embodiments, software as described herein is configured to receive data from an ECG sensor. In some embodiments, the heart-beat related data is obtained using a plurality of electrodes for recording electrical signals from the heart. In some embodiments, the heart-beat related data is obtained using two, three, four, five, six, seven, eight, nine, ten, eleven, or twelve electrodes. In some embodiments, the heart-beat related data is obtained using a 3-lead ECG sensor, a 4-lead ECG sensor, a 5-lead ECG sensor, a 6-lead ECG sensor, a 7-lead ECG sensor, an 8-lead ECG sensor, a 9-lead ECG sensor, a 10-lead ECG sensor, a 11-lead ECG sensor, or a 12-lead ECG sensor.

In some embodiments, the software application as described herein is configured to communicate with one or more different sensors, sensing devices, or other software applications or other existing monitors using hardwired or wireless transmissions. In some embodiments, the hardwired connection comprises a universal serial bus (USB) connection such as micro USB, mini USB, type B standard USB, or type A standard USB (both female and male), type C standard USB. In some embodiments, the hardwired connection comprises a Firewire connection such as 1394a (transfer speed of 400 Mbps) or 1394b (a transfer speed of 800 Mbps). In some embodiments, the hardwired connection comprises an Ethernet connection or a Lightning connection. In some embodiments, the software is configured to receive wireless transmissions using one or more protocols. For example, in some embodiments, the software is configured to send and/or receive wireless transmissions using a Zigbee transceiver, an Ultra-Wideband (UWB) transceiver, a WiFi-Direct transceiver, a Bluetooth Low Energy (BLE) transceiver, or other technologies which allow for data transfer.

In some embodiments, the software is configured to determine a pulse deficit value in a subject during one or more physiological states. In some embodiments, a physiological state is a resting state, a sleep state, or an active state. In some embodiments, a physiological state is an everyday state. A resting state generally refers to a state when the subject is awake in a neutral environment (e.g., room temperature) without having experienced any recent exertion or stimulation such as exercise or stressful stimulus. In some embodiments, the active state refers to a state where the subject is awake and has experienced recent and/or ongoing exertion or stimulation. For example, in some embodiments, the active state refers to a state of active exercise. Examples of exercise include walking, jogging, sprinting, swimming, cycling, climbing, weight training, and plyometrics. In some embodiments, exercise comes in various categories such as aerobic, anaerobic, flexibility, and balance exercises. In some embodiments, the software is configured to determine a pulse deficit value in a subject in a resting state. In some embodiments, the software is configured to determine if the subject is in a resting state by comparing sensor data to historical sensor data. For example, in some embodiments, historical sensor data is analyzed to identify periods of sleep state based on extended time periods of low heart rates relative to the overall historical heart rate. In some embodiments, historical data refers to data generated before the current data collection period. For example, current data refers to sensor data that is actively being collected and/or expanded by an apparatus as described herein to calculate heart-beat rate and pulsation rate and to determine pulse deficit value(s); data that has already been collected and analyzed to determine pulse deficit value(s) refers to historical data. In some embodiments, historical data refers to data that has been collected in the past. In such embodiments, historical data is data that has been collected at least 1 week, at least 6 days, at least 5 days, at least 4 days, at least 3 days, at least 2 days, at least 24 hours, at least 20 hours, at least 16 hours, at least 12 hours, at least 8 hours, at least 4 hours, at least 3 hours, at least 2 hours, at least 60 minutes, at least 50 minutes, at least 40 minutes, at least 30 minutes, at least 25 minutes, at least 20 minutes, at least 15 minutes, at least 10 minutes, at least 5 minutes, at least 4 minutes, at least 3 minutes, at least 2 minutes, at least 60 seconds, at least 50 seconds, at least 40 seconds, at least 30 seconds, at least 25 seconds, at least 20 seconds, at least 15 seconds, at least 10 seconds, at least 5 seconds, or at least 1 second in the past. In some embodiments, current data is any data that is not historical data.

In some embodiments, the software is configured to determine a pulse deficit value in a subject during a sleep state. Likewise, in some embodiments, historical sensor data is analyzed to identify periods of a resting state and/or active state. For instance, in some embodiments, a resting state is detected as time periods of relatively low heart rates that are higher than the heart rate during the sleep state. In some embodiments, the software is configured to determine a pulse deficit value in a subject during a resting state. In some embodiments, the active state is detected as time periods of heart rates that exceed the heart rates of the estimated sleep and/or resting states. In some embodiments, the software is configured to determine a pulse deficit value in a subject during an active state such as during exercise. In some embodiments, the software is configured to determine a pulse deficit value in a subject during an active state such as during exercise. In some embodiments, the exercise is categorized as mild, moderate, or strenuous exercise. In some embodiments, mild exercise corresponds to a heart rate that is no more than 30%, no more than 40%, no more than 50%, or no more than 60% of a maximum estimated heart rate. In some embodiments, moderate exercise corresponds to a heart rate that is at least 30%, at least 40%, at least 50%, at least 60%, at least 70%, at least 80%, or at least 85% of a maximum estimated heart rate, and/or no more than 50%, no more than 60%, no more than 70%, no more than 80%, no more than 85%, or no more than 90% of the maximum estimated heart rate. In some embodiments, intense exercise corresponds to a heart rate that is at least 60%, at least 70%, at least 80%, at least 85%, at least 90%, or at least 95% of a maximum estimated heart rate. The maximum estimated heart rate can be based on age. For example, in some embodiments, a maximum estimated heart rate is 200 bpm for a 20 year old, 195 bpm for a 25 year old, 190 bpm for a 30 year old, 185 bpm for a 35 year old, 180 bpm for a 40 year old, 175 bpm for a 45 year old, 170 bpm for a 50 year old, 165 bpm for a 55 year old, 160 bpm for a 60 year old, 155 bpm for a 65 year old, or 150 bpm for a 70 year old.

In some embodiments, the software is configured to only calculate and/or utilize the pulse deficit value(s) during a particular physiological state. In some embodiments, the software is configured to only calculate and/or utilize the pulse deficit value(s) during the resting state. In some embodiments, the software is configured to only calculate and/or utilize the pulse deficit value(s) during the sleep state. In some embodiments, the software is configured to only utilize the pulse deficit values calculated during the active state. In some embodiments, the software is configured to only calculate and/or utilize the pulse deficit value(s) during a subset of the possible physiological states. For example, in some embodiments, the software is configured to only calculate and/or utilize the pulse deficit value(s) during two physiological states selected from a resting state, a sleep state, and an active state. In some embodiments, the software is configured to calculate and/or utilize the pulse deficit values during a period of time selected or otherwise specified by a subject. For example, in some embodiments, the software is configured to receive a user command or interaction with a user interface of the apparatus as described herein indicating a period of time for calculating and/or utilizing pulse deficit value(s) (e.g., subject presses a button for initiating sensor data collection and/or analysis and presses the same button or a different button to cease sensor data collection and/or analysis). In such embodiments, allowing the subject to determine the period of time for calculating and/or utilizing pulse deficit value(s) helps narrow the analysis to relevant sensor data. For instance, a subject can provide a command to cease sensor data collection or analysis when the subject is about to undergo physical movement that can disrupt or add noise to the signals detected by the sensor(s) (e.g., physical movement can add noise to PPG sensors readings). In some embodiments, the software as described herein is configured to receive a user command or interaction indicating a change in physiological state. In some embodiments, the software as described herein is configured to detect when a user interface on the apparatus or Holter monitor receives a user interaction (e.g., subject presses a button on the interface) indicating the subject is transitioning from one state to another such as upon waking up, going to bed, about to engage in physical activity such as exercise, or upon ceasing physical activity. In such embodiments, the software as described herein is configured to incorporate the user command or interaction when determining the physiological state(s) of the subject. In some embodiments, the software as described herein is configured to categorize the periods of time that are demarcated by the transition points between physiological states obtained from the user interface.

In some embodiments, the software is integrated into the hardware of the apparatus in order to provide calculation of the pulse deficit value. In some embodiments, the software is hardware agnostic and is capable of being installed and configured to calculation pulse deficit value for a variety of hardware configurations. For example, in some embodiments, the software comprises an application programming interface that is configurable to receive input sensor data, format the data as required, perform data analysis, and output the results of the analysis according to the methods described herein. In some embodiments, the software is configurable to interface with a variety of different sensor inputs such as ECG, PPG, and impedance sensors as described herein. In some embodiments, the software is configurable to interface with a variety of inputs/outputs such as displays and can engage in communications with other computing devices and apparatuses (e.g., a Holter monitor). In some embodiments, the software is configured to calculate the pulse deficit value in real-time. In some embodiments, the software is configured to store the sensor data and the calculated pulse deficit value(s) on a memory of the apparatus. In some embodiments, the software is configured to display the number of heart-beat occurrences over a period of time on a display. In some embodiments, the software is configured to display the number of pulsation occurrences over a period of time on a display. In some embodiments, the software is configured to display the pulse deficit value(s) on a display. In some embodiments, the software is configured to transfer data (e.g. sensor data, calculated pulse deficit values, etc) to the cloud. In some embodiments, the software is configured to synchronize data with the cloud. In some embodiments, the software is configured to receive data from the cloud such as data uploaded to the cloud by other monitoring devices. In some embodiments, the software is configured to integrate data with other devices. In some embodiments, the software is configured to integrate data with other devices to continuously calculated pulse deficit values.

In some embodiments, the software is configured to generate instructions based on the predicted risk category. In some embodiments, the instructions comprise further configured to cause the processor to generate instructions based on the pulse deficit or predicted risk category. In some embodiments, the instructions comprise a personalized therapy regimen for reducing a risk of an adverse event. In some embodiments, the instructions comprise taking medication for a low predicted risk category. In some embodiments, the medication for a low predicted risk category comprises amiodorone or procainamide. In some embodiments, the medication comprises anti-arrhythmic medication. In some embodiments, the anti-arrhythmic medication comprises an agent that is classified as class Ia, class Ib, class Ic, class II, class III, class IV, or class V (Vaughan Williams classification). Class Ia agents include Quinidine, Ajmaline, Procainamide, and Disopyramide. Class Ib agents include Lidocaine, Phenytoin, Mexiletine, and Tocainide. Class Ic agents include Encainide, Flecainide, Propafenone, and Moricizine. Class II agents include Carvedilol, Propranolol, Esmolol, Timolol, Metoprolol, Atenolol, Bisoprolol, and Nebivolol. Class III agents include Amiodarone, Sotalol, Ibutilide, Dofetilide, Dronedarone, E-4031, and Vernakalant. Class IV agents include Verapamil and Diltiazem. Class V agents include Adenosine, Digoxin, and Magnesium Sulfate. In some embodiments, the instructions comprise applying an anti-arrhythmic strategy for a high predicted risk category. In some embodiments, the anti-arrhythmic strategy comprises cardioversion or ablation. In some embodiments, the instructions comprise identification of drugs that are not recommended (or advised against). For example, in some embodiments, instructions recommend against taking drugs that are predicted to be ineffective for treating the predicted risk category. In some embodiments, the instructions comprise increased visitation to and/or monitoring by a cardiologist or heart specialist. In some embodiments, the instructions comprise using anti-arrhythmic medication to adjust the pulse deficit.

In some embodiments, the software is configured to generate a summary of the sensor data and/or analysis of the sensor data. In some embodiments, the software is configured to send the summary to a display of the apparatus or system to be viewed by the subject. In some embodiments, the software is configured to send the summary to another device such as, for example, a server on a network, a communication device of the subject (e.g., the subject's phone), or a computing device of a healthcare provider (e.g., the subject's doctor). In some embodiments, the summary comprises historical data such as sensor data, heart-beat rate, pulse rate, delta value or difference between heart-beat and pulse rates, and pulse deficit value(s). In some embodiments, the summary comprises a comparison between historical data and current data. For example, in one embodiment, the summary provides a historical pulse deficit value from a week ago alongside a current pulse deficit value and a difference between the historical pulse deficit value and the current pulse deficit value. In some embodiments, the summary comprises instructions based on the predicted risk category.

In some embodiments, the software is configured to compare sensor data and/or analysis of the sensor data for the subject to reference data or information such as, for example, exemplary data expected for Atrial Fibrillation patients. In some embodiments, the exemplary data is generated to correspond to the sensor readings, heart-beat rate, pulsation rate, and pulse deficit value(s) expected for an Atrial Fibrillation patient. In some embodiments, the comparison utilizes exemplary data for Atrial Fibrillation patients as a reference to determine one or more of the subject's heart-beat rate, pulsation rate, difference or delta between the heart-beat rate and pulsation rate, and pulse deficit value(s) relative to expected values in an Atrial Fibrillation patient. In some embodiments, the comparison determines one or more of the subject's heart-beat rate, pulsation rate, difference or delta between the heart-beat rate and pulsation rate, and pulse deficit value(s) as a percentile within a population of Atrial Fibrillation patients. For example, the subject may have a pulse deficit value that is in the 90^{th} percentile of the Atrial Fibrillation patient, indicating the subject has a higher than average pulse deficit value. In some embodiments, the software is configured to provide the comparison to an end user such as the subject or a healthcare provider responsible for the subject. In such embodiments, the comparison can be shown on a display of the apparatus and/or sent to a server, communication device, or other computing device. In some embodiments, the comparison is provided in the summary as described herein.

### Methods for Determining a Presence of a Pulse Deficit in a Subject

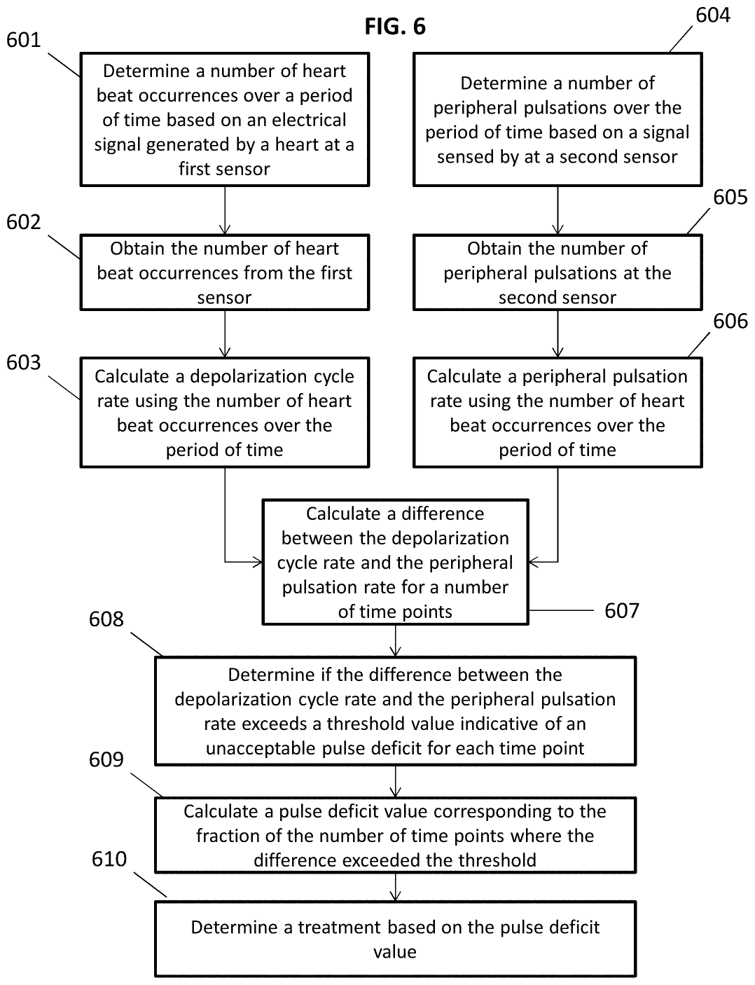

In some embodiments, described herein are methods for determining a presence of a pulse deficit in a subject. The methods comprise steps that can be performed by any of the systems, apparatuses, and software described herein. As shown in FIG. 6, a non-limiting exemplary method for a calculating pulse deficit value can comprise the following exemplary steps: In a step 601, apparatus or system detects a number of heart beat occurrences over a period of time based on an electrical signal generated by a heart at a first sensor. For example, the period of time can be at least 1 minute. The heart beat occurrences are measured as a time series of beats per minute. In a step 602, the apparatus or system obtains the number of heart beat occurrences from the first sensor. In a step 603, the apparatus or system calculates a depolarization cycle rate using the number of heart beat occurrences over the period of time. In parallel to the steps involving the first sensor, in step 604, the apparatus or system determines a number of peripheral pulsations over the period of time based on a signal sensed by at a second sensor. For example, the peripheral pulsations are measured as a time series of beats per minute. In step 605, the apparatus or system obtains the number of peripheral pulsations at the second sensor. In step 606, the apparatus or system calculates a peripheral pulsation rate using the number of heart beat occurrences over the period of time. Next, in step 607, the apparatus or system calculates a difference between the depolarization cycle rate and the peripheral pulsation rate for a number of time points. In step 608, the apparatus or system determines if the difference between the depolarization cycle rate and the peripheral pulsation rate exceeds a threshold value indicative of an unacceptable pulse deficit for each time point. In step 609, the apparatus or system calculates a pulse deficit value corresponding to the fraction of the number of time points where the difference exceeded the threshold. In step 610, optionally, the apparatus or system determines a treatment based on the pulse deficit value.

In some embodiments, a pulse deficit (e.g., an unacceptable pulse deficit) is determined to be present when any difference exists between a number of heart-beats and a number of peripheral pulsations over a period of time. In some embodiments, a pulse deficit is determined to be present when a difference between a number of heart-beats and a number of peripheral pulsations exceeds a threshold value. In some embodiments, a threshold value is equal to 1. In some embodiments, a threshold is equal to 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more. In some embodiments, a threshold is equal to at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30. In some embodiments, a threshold is equal to no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 21, no more than 22, no more than 23, no more than 24, no more than 25, no more than 26, no more than 27, no more than 28, no more than 29, or no more than 30.

In some embodiments, a pulse deficit (e.g., an unacceptable pulse deficit) is determined to be present when a difference exists between a number of heart beats and a number of peripheral pulsations over a period of time and an additional parameter is present (or multiple additional parameters are present). For example, in some embodiments, an additional parameter comprises a variation of a number of heart-beats from a baseline, wherein a pulse deficit is determined to be present when a difference exists between a number of heart beats and a number of peripheral pulsations over a period of time and a number of heart-beats varies from a baseline value. In some embodiments, the baseline value is a resting heart rate of the subject. In some embodiments, the baseline value is an arbitrary value (e.g., a population average resting heart rate). In some embodiments, the additional parameter comprises an age of a subject. In some embodiments, the additional parameter comprises a sex or gender of a subject. In some embodiments, the additional parameter comprises demographic information such as race, ethnicity, gender, age, education, profession, occupation, income level, marital status, or any combination thereof. In some embodiments, the additional parameter comprises health information such as coronary heart disease, high blood pressure, diabetes, smoking tobacco, drinking alcohol, obesity, BMI (e.g., high BMI), or any combination thereof.

In some embodiments, the data collected from the at least one ECG sensor (first sensor) is converted into a depolarization cycle rate, while the data collected from the at least one pulse sensor (second sensor) is converted into a pulsation rate. At each measured point in time, a difference between the depolarization cycle rate and the pulsation rate (the “delta value”) is calculated to produce a separate time series. In some embodiments, the delta value is determined as a whole number difference between the depolarization cycle rate and the pulsation rate. For example, in some embodiments, a whole number difference for a delta value between depolarization cycle rate and pulsation rate is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. Alternatively, in some embodiments, the delta value is determined as a percentage difference between the depolarization cycle rate and the pulsation rate. In some embodiments, the percentage difference is calculated as the difference between the depolarization cycle rate and pulsation rate divided by the depolarization cycle rate. Alternatively, in some embodiments, the delta value is calculated as the difference between the depolarization cycle rate and pulsation rate divided by the pulsation rate. Dividing by the depolarization cycle rate, which is typically a larger value than pulsation rate in subjects having Atrial Fibrillation, should generate a relatively smaller percentile difference for the delta value compared to dividing by the pulsation rate. In some embodiments, a percentage difference for the delta value between depolarization cycle rate and pulsation rate is from 0% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, or from 90% to 95%. Over the entire measured time period, the number of measured points in time where the delta value exceeded a set threshold value indicative of unacceptable pulse deficit is counted and calculated as a fraction of the total measured points in time for the entire measured time period. In some embodiments, this calculated fraction is utilized as a pulse deficit value.

In some embodiments, the calculated fraction is supplemented with other calculations in producing a pulse deficit value. One embodiment of a supplemental calculation is a histogram-based analysis. This analysis is executed by generating a histogram of depolarization cycle rate, generating a histogram of pulsation rate, and calculating a distance between these two generated histograms. The distance may be calculated in one or more of the following manners: Histogram intersection, Canberra distance, cosine distance, and Hellinger distance. In some embodiments, Histogram intersection is utilized to calculate the similarity of the discrete probability distributions or histograms of depolarization cycle rate and pulsation rate, which is represented as a value between 0 (no similarity) and 1 (identical). In some embodiments, Canberra distance is utilized to calculate the sum of a series of fraction differences between the coordinates of depolarization cycle rate and pulsation rate. In some embodiments, cosine distance is utilized to calculate a measure of distance between two non-zero vectors by measuring the cosine of the angle between them. In some embodiments, Helldinger distance is utilized to measure the similarity between the probability distributions for depolarization cycle rate and pulsation rate. The aforementioned methods are provided as non-limiting embodiments, and other methods for calculating the distance between two histograms are contemplated in the present disclosure.

In some embodiments, the pulse deficit value is calculated using a supplemental calculation such as a percentile-based analysis. This is also a non-time-aligned comparison between the depolarization cycle rate and the pulsation rate, which is optionally utilized in situations of consistently higher monitor readouts. For example, in some embodiments, the percentile-based analysis is applied when the depolarization cycle rate, the pulsation rate, or both are indicative of consistently higher monitor readouts. In some embodiments, high monitor readout is indicated by at least one of a high depolarization cycle rate and a high pulsation rate of at least 30, 40, 50, 60, 70, 80, 90, 100, 110, 120, 130, 140, 150, 160, 170, 180, 190, 200, 210, 220, 230, or 240 beats per minute. In some embodiments, the high monitor readout is determined by reference to a normal readout range (e.g., 40-80 resting bpm). Alternatively, in some embodiments, the high monitor readout is determined by reference to the range of historical readout rates for the subject. As an example, a subject whose historical readout rates falls within the 40-80 bpm range may be determined as having a high monitor readout when the subject's measured monitor readout exceeds this range. In some embodiments, the high monitor readout is determined when the subject's measured rate exceeds the historical rate range by a minimum amount. For instance, in some embodiments, the monitor readout is determined to be high when the subject's measured rate (e.g., depolarization rate and/or pulsation rate) exceeds the historical range by at least 5 bpm, 10 bpm, 15 bpm, 20 bpm, 25 bpm, 30 bpm, 35 bpm, 40 bpm, 45 bpm, or 50 bpm. In some embodiments, consistently high monitor readout is indicated by the monitor readout exceeding the historical range for at least a minimum period of time. In such embodiments, the period of time refers to any of the various ranges and values described throughout the present disclosure. The analysis is executed by calculating one or more selected percentiles for both the depolarization cycle rate and the pulsation rate, such as the 5%, 10%, 15%, 20%, 25%, 30%, 35%, 40%, 45%, 50%, 55%, 60%, 65%, 70%, 75%, 80%, 85%, 90%, and/or 95% percentiles, and calculating the difference, or delta, for each selected percentile. In some embodiments, one or more of these supplemental calculations is utilized as a supplemental calculation in producing a pulse deficit value. In some embodiments, at least one, two, three, four, or five of these supplemental calculations are used in producing a pulse deficit value. It will be understood by those of ordinary skill in the art that various changes may be made and equivalents may be substituted for elements without departing from the scope of the invention. In addition, many modifications can be made to adapt a particular feature or material to the teachings of the invention without departing from the scope thereof. Therefore, it is intended that the invention not be limited to the particular embodiments disclosed, but that the invention will include all embodiments falling within the scope of the claims.

In some embodiments, the methods described herein allow risk stratification of an Atrial Fibrillation subject based on the calculated pulse deficit value. In some embodiments, the risk stratification comprises generating a predicted risk of an adverse event. In some embodiments, the adverse event is a major adverse cardiovascular event (MACE) and can include myocardial infarction, stroke, cardiovascular death, or a combination thereof. In some embodiments, the risk stratification is performed for a defined period of time such as, for example, about 1 month, about 2 months, about 3 months, about 4 months, about 5 months, or about 6 months.

In some embodiments, the risk stratification is performed using a risk stratification classifier. In some embodiments, the classifier comprises an algorithm that receives an input (e.g., pulse deficit value) and generates as output a predicted risk of an adverse event. Various algorithms can be used to generate models that predict a risk of an adverse event. The features selected for classification can include the calculated difference or delta between the heart-beat rate and the peripheral pulsation rate. The heart-beat rate and peripheral pulsation rates, and their delta values can be calculated as described herein.

### Systems for Determining a Presence of a Pulse Deficit in a Subject

In some embodiments, a system as described herein is configured to determine a presence of a pulse deficit in a subject. In some embodiments, a system as described herein comprises an apparatus including sensors for determining the number of heart beat occurrences and peripheral pulsations over a period of time as described herein. In some embodiments, a system as described herein comprises a network element for communicating with a server. In some embodiments, a system as described herein comprises a server. In some embodiments, the system is configured to upload to and/or download data from the server. In some embodiments, the server is configured to store sensor data, pulse deficit value(s), and/or other information for the subject. In some embodiments, the server is configured to store historical data (e.g., past sensor data and/or pulse deficit value(s)) for the subject. In some embodiments, the server is configured to backup data from the system or apparatus. In some embodiments, a system as described herein is configured to perform any of the methods described herein.

In some embodiments, a system as described herein is configured to determine a presence of a pulse deficit in a subject, the system comprising a network element communicating with a server on a network and an apparatus, the apparatus comprising: a first sensor configured to determine a number of heart beat occurrences over a period of time based on an electrical signal generated by a heart and sensed by the first sensor; a second sensor configured to determine a number of peripheral pulsations over the period of time based on a signal sensed by the second sensor; a processor; a non-transitory computer-readable medium including instructions executable by the processor and configured to cause the processor to: receive the number of heart beat occurrences over the period of time; receive the number of pulsation occurrences over the period of time; and identify the presence of the pulse deficit which comprises a numerical difference between the number of heart beat occurrences over the period of time and the number of pulsation occurrences over the period of time.

In some embodiments, the system or apparatus is configured to encrypt data. In some embodiments, data on the server is encrypted. In some embodiments, the system or apparatus comprises a data storage unit or memory for storing data. In some embodiments, data encryption is carried out using Advanced Encryption Standard (AES). In some embodiments, data encryption is carried out using 128-bit, 192-bit, or 256-bit AES encryption. In some embodiments, data encryption comprises full-disk encryption of the data storage unit (e.g., encrypting the entire hard drive on a server or apparatus). In some embodiments, data encryption comprises virtual disk encryption (e.g., encrypting a folder containing sensor data files for a subject). In some embodiments, data encryption comprises file encryption (e.g., encrypting sensor data files for a subject). In some embodiments, data that is transmitted or otherwise communicated between the system or apparatus and other devices or servers is encrypted during transit. In some embodiments, wireless communications between the system or apparatus and other devices or servers is encrypted. As an example, an apparatus that is integrated with a Holter monitor sends and/or receives data wirelessly using an encrypted data channel. In some embodiments, data in transit is encrypted using a Secure Sockets Layer (SSL). In some embodiments, access to data stored on the system or apparatus as described herein requires user authentication. In some embodiments, access to data stored on the server as described herein requires user authentication.

An apparatus as described herein comprises a digital processing device that includes one or more hardware central processing units (CPUs) or general purpose graphics processing units (GPGPUs) that carry out the device's functions. The digital processing device further comprises an operating system configured to perform executable instructions. The digital processing device is optionally connected to a computer network. The digital processing device is optionally connected to the Internet such that it accesses the World Wide Web. The digital processing device is optionally connected to a cloud computing infrastructure. Suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein.

Typically, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing.

A digital processing device as described herein either includes or is operatively coupled to a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

A system or method as described herein can be used to generate a pulse deficit value which may then be used to determine whether a subject value falls within or outside of a threshold value. In addition, in some embodiments, a system or method as described herein generates a database as containing or comprising one or more pulse deficit values. In some embodiments, a database herein provides a relative risk of presence/absence of a status (outcome) associated with one or more pulse deficit values that fall either within or outside of a threshold value.

Some embodiments of the systems described herein are computer based systems. These embodiments include a CPU including a processor and memory which may be in the form of a non-transitory computer-readable storage medium. These system embodiments further include software that is typically stored in memory (such as in the form of a non-transitory computer-readable storage medium) where the software is configured to cause the processor to carry out a function. Software embodiments incorporated into the systems described herein contain one or more modules.

Some of the software embodiments described herein are configured to cause a processor to: receive the number of heart-beat occurrences over the period of time; receive the number of pulsation occurrences over the period of time; and identify the presence of the pulse deficit which comprises a numerical difference between the number of heart-beat occurrences over the period of time and the number of pulsation occurrences over the period of time.

In various embodiments, an apparatus comprises a computing device or component such as a digital processing device. In some of the embodiments described herein, a digital processing device includes a display to send visual information to a user. Non-limiting examples of displays suitable for use with the systems and methods described herein include a liquid crystal display (LCD), a thin film transistor liquid crystal display (TFT-LCD), an organic light emitting diode (OLED) display, an OLED display, an active-matrix OLED (AMOLED) display, or a plasma display.

A digital processing device, in some of the embodiments described herein includes an input device to receive information from a user. Non-limiting examples of input devices suitable for use with the systems and methods described herein include a keyboard, a mouse, trackball, track pad, or stylus. In some embodiments, the input device is a touch screen or a multi-touch screen.

The systems and methods described herein typically include one or more non-transitory computer-readable storage media encoded with a program including instructions executable by the operating system of an optionally networked digital processing device. In some embodiments of the systems and methods described herein, the non-transitory storage medium is a component of a digital processing device that is a component of a system or is utilized in a method. In still further embodiments, a computer-readable storage medium is optionally removable from a digital processing device. In some embodiments, a computer-readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

Typically the systems and methods described herein include at least one computer program, or use of the same. A computer program includes a sequence of instructions, executable in the digital processing device's CPU, written to perform a specified task. Computer-readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages. The functionality of the computer-readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

Typically, the systems and methods described herein include and/or utilize one or more databases. In view of the disclosure provided herein, those of skill in the art will recognize that many databases are suitable for storage and retrieval of baseline datasets, files, file systems, objects, systems of objects, as well as data structures and other types of information described herein. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

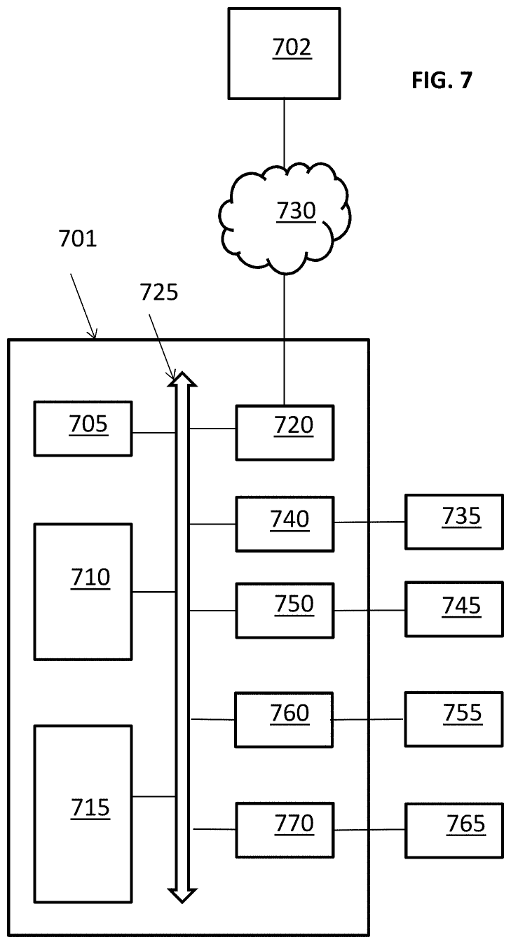

FIG. 7 shows an exemplary embodiment of a system as described herein comprising an apparatus such as a digital processing device 701. The digital processing device 701 includes a software application configured to monitor the cardiovascular health of an individual by, for example, determining a presence of a pulse deficit. The digital processing device 701 may include a central processing unit (CPU, also “processor” and “computer processor” herein) 705, which can be a single core or multi-core processor, or a plurality of processors for parallel processing. The digital processing device 701 also includes either memory or a memory location 710 (e.g., random-access memory, read-only memory, flash memory), electronic storage unit 715 (e.g., hard disk), communication interface 720 (e.g., network adapter, network interface) for communicating with one or more other systems, and peripheral devices, such as cache. The peripheral devices can include storage device(s) or storage medium 765 which communicate with the rest of the device via a storage interface 770. The memory 710, storage unit 715, interface 720 and peripheral devices are configured to communicate with the CPU 705 through a communication bus 725, such as a motherboard. The digital processing device 701 can be operatively coupled to a computer network (“network”) 730 with the aid of the communication interface 720. The network 730 can comprise the Internet. The network 730 can be a telecommunication and/or data network.

The digital processing device 701 includes input device(s) 745 to receive information from a user, the input device(s) in communication with other elements of the device via an input interface 750. The digital processing device 701 can include output device(s) 755 that communicates to other elements of the device via an output interface 760.

The CPU 705 is configured to execute machine-readable instructions embodied in a software application or module. The instructions may be stored in a memory location, such as the memory 710. The memory 710 may include various components (e.g., machine readable media) including, but not limited to, a random access memory component (e.g., RAM) (e.g., a static RAM “SRAM”, a dynamic RAM “DRAM, etc.), or a read-only component (e.g., ROM). The memory 710 can also include a basic input/output system (BIOS), including basic routines that help to transfer information between elements within the digital processing device, such as during device start-up, may be stored in the memory 710.

The storage unit 715 can be configured to store files, such as health or risk parameter data, e.g., individual health or risk parameter values, health or risk parameter value maps, and value groups. The storage unit 715 can also be used to store operating system, application programs, and the like. Optionally, storage unit 715 may be removably interfaced with the digital processing device (e.g., via an external port connector (not shown)) and/or via a storage unit interface. Software may reside, completely or partially, within a computer-readable storage medium within or outside of the storage unit 715. In another example, software may reside, completely or partially, within processor(s) 705.

Information and data can be displayed to a user through a display 735. The display is connected to the bus 725 via an interface 740, and transport of data between the display other elements of the device 701 can be controlled via the interface 740.

Methods as described herein can be implemented by way of machine (e.g., computer processor) executable code stored on an electronic storage location of the digital processing device 701, such as, for example, on the memory 710 or electronic storage unit 715. The machine executable or machine readable code can be provided in the form of a software application or software module. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

In some embodiments, a remote device 702 is configured to communicate with the digital processing device 701, and may comprise any mobile computing device, non-limiting examples of which include a tablet computer, laptop computer, smartphone, or smartwatch. For example, in some embodiments, the remote device 702 is a smartphone of the user that is configured to receive information from the digital processing device 701 of the apparatus or system described herein in which the information can include a summary, sensor data, pulse deficit value(s), or other data. In some embodiments, the remote device 702 is a server on the network configured to send and/or receive data from the apparatus or system described herein.

Some embodiments of the systems and methods described herein are configured to generate a database containing or comprising of one or more pulse deficit values and/or threshold value. A database, as described herein, is configured to function as, for example, a lookup table for healthcare providers, other medical industry professionals and/or other end users. In these embodiments of the systems and methods described herein, pulse deficit values are presented in a database so that a user is able to, for example, identify whether a parameter of a specific subject falls within or outside of a threshold value. In some embodiments, the database is stored on a server on the network. In some embodiments the database is stored locally on the apparatus (e.g., the monitor component of the apparatus). In some embodiments, the database is stored locally with data backup provided by a server.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A system configured to determine a presence of a pulse deficit in a subject, the system comprising:
(a) a first sensor configured to determine a number of heart-beat occurrences over a period of time based on an electrical signal generated by a heart and sensed by the first sensor;
(b) a second sensor configured to determine a number of peripheral pulsations over the period of time based on a signal sensed by the second sensor;
(c) a processor;
(d) a network element configured to communicate with a network; and
(e) a non-transitory computer-readable medium including instructions executable by the processor and configured to cause the processor to:
(i) receive the number of heart-beat occurrences over the period of time;
(ii) receive the number of pulsation occurrences over the period of time; and
(iii) identify the presence of the pulse deficit which comprises a numerical difference between the number of heart-beat occurrences over the period of time and the number of pulsation occurrences over the period of time.

2. The system of claim 1, comprising a risk stratification classifier configured to assess the risk of an adverse health event occurring to the subject based on the presence of an unacceptable pulse deficit.

3. The system of claim 2, wherein a degree of risk of the adverse event occurring corresponds directly to the degree of the numerical difference between the number of heart-beat occurrences over the period of time and the number of pulsation occurrences over the period of time.

4. The system of claim 2, wherein the risk stratification classifier generates a predicted risk category indicative of the risk of an adverse health event.

5. The system of claim 4, wherein the non-transitory computer-readable medium is further configured to cause the processor to:
(a) determine a heart rate histogram and a pulse rate histogram;
(b) calculate a cosine distance between the heart rate histogram and the pulse rate histogram; and
(c) input the cosine distance into the risk stratification classifier to generate the predicted risk category.

6. The system of claim 4, wherein the non-transitory computer-readable medium is further configured to cause the processor to:
(a) determine the heart rate and the pulse rate for at least two percentiles for a plurality of time points;
(b) calculate delta values between the heart rate and the pulse rate for at least two percentiles; and
(c) input the delta values for at least two percentiles into the risk stratification classifier to generate the predicted risk category.

7. The system of claim 6, wherein the at least two percentiles comprise about 25%, about 50%, and about 75%.

8. The system of claim 1, wherein the first sensor comprises an electrocardiogram (ECG) sensor and wherein the second sensor comprises a photoplethysmographic (PPG) pulse sensor, a bioimpedance plethysmograph, an accelerometer, or a pressure sensor.

9. The system of claim 1, further comprising a display for showing at least one of the heart rate, the pulse rate, the pulse deficit value, and the predicted risk category.

10. The system of claim 4, wherein the non-transitory computer-readable medium is further configured to cause the processor to generate instructions based on the pulse deficit value or predicted risk category, wherein the instructions comprise a personalized therapy regimen for reducing a risk of an adverse event.

11. A computer-implemented method for determining a presence of a pulse deficit in a subject, the method comprising:
(a) determining a number of heart-beat occurrences over a period of time based on an electrical signal generated by a heart and sensed by a first sensor;
(b) determining a number of peripheral pulsations over the period of time based on a signal sensed by a second sensor; and
(c) identifying the presence of the pulse deficit which comprises a numerical difference between the number of heart-beat occurrences over the period of time and the number of pulsation occurrences over the period of time.

12. The method of claim 11, further comprising providing a risk stratification classifier configured to assess the risk of an adverse health event occurring to the subject based on the presence of an unacceptable pulse deficit.

13. The method of claim 12, wherein a degree of risk of the adverse event occurring corresponds directly to the degree of the numerical difference between the number of heart-beat occurrences over the period of time and the number of pulsation occurrences over the period of time.

14. The method of claim 12, wherein the risk stratification classifier generates a predicted risk category indicative of the risk of an adverse health event.

15. The method of claim 14, wherein the method further comprises:
(a) determining a heart rate histogram and a pulse rate histogram;
(b) calculating a cosine distance between the heart rate histogram and the pulse rate histogram; and
(c) inputting the cosine distance into the risk stratification classifier to generate the predicted risk category.

16. The method of claim 14, wherein the method further comprises:
(a) determining the heart rate and the pulse rate for at least two percentiles for a plurality of time points;
(b) calculating delta values between the heart rate and the pulse rate for at least two percentiles; and
(c) inputting the delta values for at least two percentiles into the risk stratification classifier to generate the predicted risk category.

17. The method of claim 16, wherein the at least two percentiles comprise about 25%, about 50%, and about 75%.

18. The method of claim 11, wherein the first sensor comprises an electrocardiogram (ECG) sensor and wherein the second sensor comprises a photoplethysmographic (PPG) pulse sensor, a bioimpedance plethysmograph, a gyroscope, an accelerometer, or a pressure sensor.

19. The method of claim 14, further comprising showing at least one of the heart rate, the pulse rate, the pulse deficit value, and the predicted risk category on a display.

20. The method of claim 14, further comprising generating instructions based on the pulse deficit value or predicted risk category, wherein the instructions comprise a personalized therapy regimen for reducing a risk of an adverse event.
